(19) United States
(12) Patent Application Publication
(10) Pub. No.: US 2008/0004313 A1
(43) Pub. Date: Jan. 3, 2008

(54) Preparation of crystalline polymorphs of rimonabant hydrochloride

(75) Inventors: Hui Min He Huang, Northbrough, MA (US); Cai Gu Huang, Northbrough, MA (US)

Correspondence Address: Mai De Ltd., P. O. Box 193, Northborough, MA, 01532, US

(73) Assignee: Mai De Ltd.

(21) Appl. No.: 11/818,986

(22) Filed: Jun. 18, 2007

Related U.S. Application Data
(60) Provisional application No. 60/816,670, filed on Jun. 28, 2006.

(51) Int. Cl. A61K 31/454; C07D 401/12

(52) U.S. Cl. 514/326; 546/211

(57) ABSTRACT

This invention relates to three novel solvate forms of crystalline rimonabant hydrochloride (Form A, B, and C) and two novel crystalline polymorphic forms of rimonabant hydrochloride anhydrous (Form D and E), to processes for preparing such polymorphic forms or solvate forms, to pharmaceutical compositions containing them and to the use of them in medicine.

This application claims the benefit of priority to U.S. Provisional Patent Application Ser. No. 60/816,670 filed on Jun. 28, 2006, the entire disclosure of which is hereby incorporated by reference.

### FIELD OF THE INVENTION

This invention relates to two novel polymorphic forms of crystalline rimonabant hydrochloride and three novel crystalline forms of rimonbant hydrochloride solvates, to processes for preparing such polymorphic forms or solvate forms, to pharmaceutical compositions containing them and to the use of them in medicine.

### BACKGROUND OF THE INVENTION

Rimonabant hydrochloride, its chemical name is N-piperidino-5-(4-chlorophenyl)-1-(2,4-dichlorophenyl)-4-methyl-3-pyrazolecarboxamide hydrochloride, has the following structural formula:

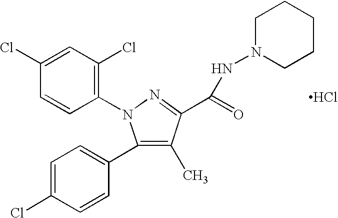

Rimonabant and rimonabant acid addition salts such as rimonabant hydrochloride are antagonists of the CB₁ cannabinoid receptors, which were first described in U.S. Pat. No. 5,624,941. Example 196 of this patent describes the preparation of rimonabant hydrochloride, which was obtained by the reaction of rimonabant (free base) with a saturated solution of gaseous HCl in ether solvent. At pH=1, the product was precipitated, filtered off, washed with ether and dried under vacuum to give the expected product of rimonabant hydrochloride with melting point=224° C. (decomposition). This patent and other prior arts do not disclose or refer to, or even suggest the possible existence of crystalline polymorphs of rimonabant hydrochloride or its solvates. Each crystalline polymorph or solvate of a given compound may display different melting point, hygroscopicity, stability, solubility and dissolution rate, crystallinity, bioavailability and handling characteristics, which are among the numerous properties that need to be considered in preparing medicament that can be effectively administered. Accordingly, there is a going need to prepare crystalline polymorphs or solvates that are both physiologically acceptable and suitable for preparing reproducible pharmaceutical formulations. The crystalline polymorphs of rimonabant hydrochloride or its solvates in the present invention help fulfill this and other needs.

### SUMMARY OF THE INVENTION

The applicant has now surprisingly discovered that rimonabant hydrochloride can exist in three crystalline solvate forms (designed as Form A, Form B, Form C) and two novel crystalline polymorphic forms (designed as Form D and Form E), which differ from each other in their stability, physicochemical properties, in their spectral characteristics and in their processes of preparations. In addition, novel crystalline polymorphs or solvates of rimonabant hydrochloride discovered by applicant have useful and satisfactory pharmaceutical properties, and are particularly suitable for bulk preparation, handling and formulation advantages, and can be prepared by an efficient, economic and reproducible processes suitable to large-scale production in industry.

Therefore, as a first aspect, the present invention provides novel crystalline ethanol solvate of rimonabant hydrochloride or rimonabant hydrochloride ethanolate (designed as Form A).

According to another aspect, the present invention provides novel crystalline dichloromethane solvate of rimonabant hydrochloride or rimonabant hydrochloride dichloromethanate (designed as Form C) with a molar ratio of 3:1.

According to a further aspect, the present invention provides novel crystalline hemi-isopropanol solvate of rimonabant hydrochloride or rimonabant hydrochloride hemi-isopropanolate (designed as Form C) with a molar ratio of 2:1.

According to a still aspect, the present invention provides a novel anhydrous crystalline polymorph of rimonabant hydrochloride (designed as Form D).

According to a still another aspect, the present invention provides a novel anhydrous crystalline polymorph of rimonabant hydrochloride (designed as Form E).

In another aspect, the present invention provides processes for preparing polymorphic forms or solvates (Form A, Form B, Form C, Form D and Form E) of crystalline rimonabant hydrochloride.

Accordingly, the present invention also provides a pharmaceutical composition comprising any of polymorphic forms or solvates (Form A, Form B, Form C, Form D or Form E) of crystalline rimonabant hydrochloride as described above and one or more pharmaceutically acceptable diluents or carriers and, optionally, one or more other physiologically active agents.

In a still aspect, the present invention provides the use of polymorphic forms or solvates (Form A, Form B, Form C, Form D or Form E) of crystalline rimonabant hydrochloride for the treatment and/or prophylaxis of a disease or disorder, particularly obesity and related metabolic risk factors, tobacco dependence and certain complications thereof, in which an antagonist of the GB₁ cannabinoid receptor in involved.

### BRIEF DESCRIPTION OF THE ACCOMPANYING DRAWINGS

FIG. 1: X-ray powder diffraction pattern of Form A of rimonabant hydrochloride.

FIG. 2: Differential scanning calorimetry (DSC) thermogram of Form A of rimonabant hydrochloride.

FIG. 3: Infrared spectrum of Form A of rimonabant hydrochloride.

FIG. 4: Thermogravimetric analysis (TGA) thermogram of Form A of rimonabant hydrochloride.

FIG. 5: X-ray powder diffraction pattern of Form B of rimonabant hydrochloride.

FIG. 6: Differential scanning calorimetry (DSC) thermogram of Form B of rimonabant hydrochloride.

FIG. 7: Infrared spectrum of Form B of rimonabant hydrochloride.

FIG. 8: Thermogravimetric analysis (TGA) thermogram of Form B of rimonabant hydrochloride.

FIG. 9: X-ray powder diffraction pattern of Form C of rimonabant hydrochloride.

FIG. 10: Differential scanning calorimetry (DSC) thermogram of Form C of rimonabant hydrochloride.

FIG. 11: Infrared spectrum of Form C of rimonabant hydrochloride.

FIG. 12: Thermogravimetric analysis (TGA) thermogram of Form C of rimonabant hydrochloride.

FIG. 13: X-ray powder diffraction pattern of Form D of rimonabant hydrochloride.

FIG. 14: Differential scanning calorimetry (DSC) thermogram of Form D of rimonabant hydrochloride.

FIG. 15: Infrared spectrum of Form D of rimonabant hydrochloride.

FIG. 16: Thermogravimetric analysis (TGA) thermogram of Form D of rimonabant hydrochloride.

FIG. 17: X-ray powder diffraction pattern of Form E of rimonabant hydrochloride.

FIG. 18: Differential scanning calorimetry (DSC) thermogram of Form E of rimonabant hydrochloride.

FIG. 19: Infrared spectrum of Form E of rimonabant hydrochloride.

FIG. 20: Thermogravimetric analysis (TGA) thermogram of Form E of rimonabant hydrochloride

### DETAILED DESCRIPTION OF THE INVENTION

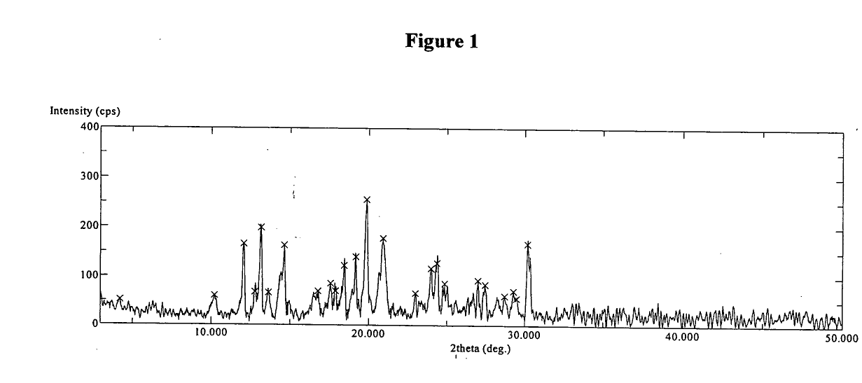

Accordingly, the present invention provides a novel and well-defined crystalline ethanol solvate of rimonabant hydrochloride, also termed as crystalline rimonabant hydrochloride ethanolate, designed hereinafter as Form A of rimonabant hydrochloride in this application, having an X-ray powder diffraction pattern, or substantially the same X-ray powder diffraction pattern, as shown in FIG. 1.

More particularly, crystalline rimonabant hydrochloride ethanolate according to the present invention can be characterized as having an X-ray diffraction pattern with characteristic peaks (expressed in degrees 2θ, accuracy is +/−0.2 degree): 10.20, 12.04, 12.76, 13.14, 13.62, 14.62, 16.76, 17.56, 17.88, 18.44, 19.18, 19.86, 20.90, 22.96, 23.96, 24.34, 24.84, 27.00, 27.46, 28.70, 29.28, 29.48 and 30.18.

Characterizing data for crystalline rimonabant hydrochloride ethanolate according to the present invention as obtained by X-ray powder diffraction pattern is shown in FIG. 1 and Table 1.

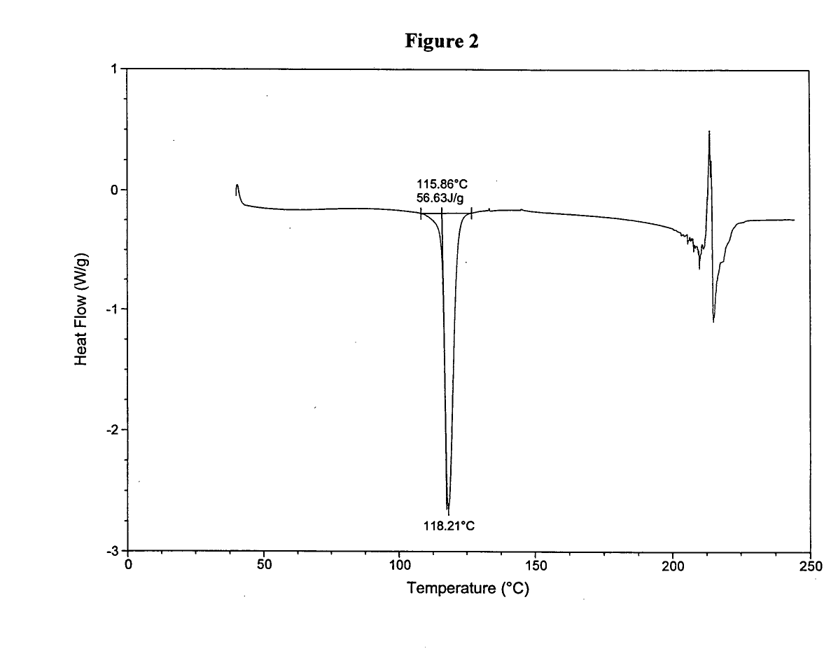

Further characterizing data for crystalline rimonabant hydrochloride ethanolate according to the present invention as obtained by differential scanning calorimetry (DSC) is shown in FIG. 2. The freshly prepared crystalline rimonabant hydrochloride ethanolate provides a characteristic peak at about 112-114° C. in its DSC thermogram. After about a month standing at ambient temperature, it provides a characteristic peak at about 118° C. in its DSC thermogram. The characteristic peak at 112-118° C. in its DSC thermogram is due to the desolvation of ethanol solvent from crystalline rimonabant hydrochloride ethanolate upon heating.

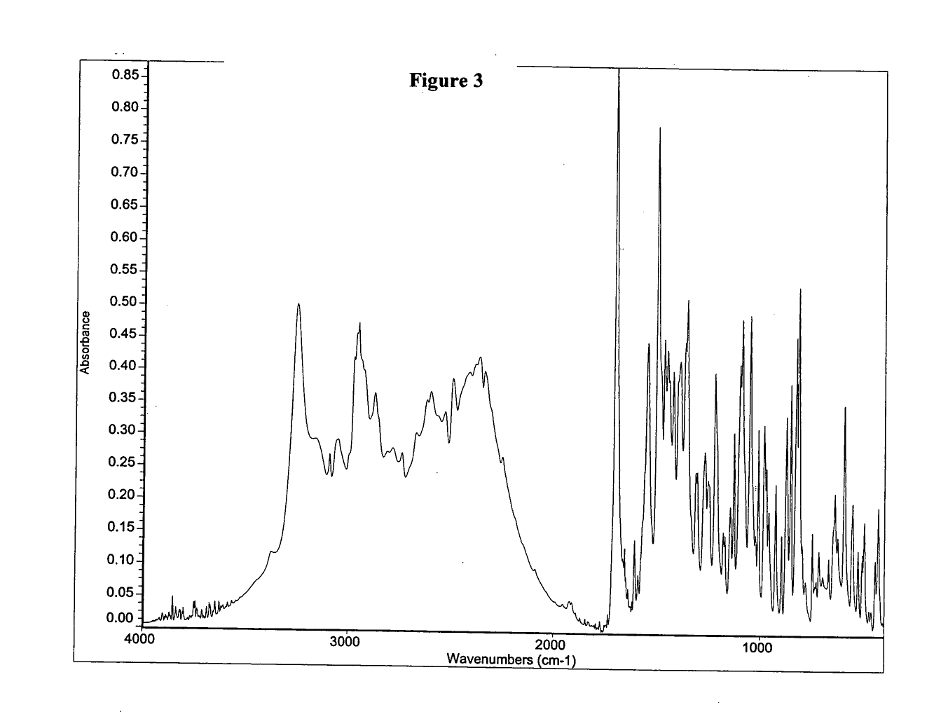

Still further characterizing data for crystalline rimonabant hydrochloride ethanolate according to the present invention as obtained by infrared spectroscopy (KBr adsorption method) is shown in FIG. 3, and it provides characteristic absorbance bands at 3254, 3093, 3051, 2953, 2873, 2602, 2495, 2365, 1700, 1605, 1544, 1496, 1462, 1448, 1419, 1385, 1352, 1310, 1266, 1250, 1217, 1176, 1143, 1125, 1099, 1088, 1049, 1010, 981, 970, 959, 926, 896, 874, 853, 825, 814, 747, 715, 668, 639, 593, 552, 525, 496, 442 and 428 cm⁻¹. Absorption band 3254 cm⁻¹ clearly indicates the presence of a hydroxyl group of ethanol from crystalline rimonabant hydrochloride ethanolate.

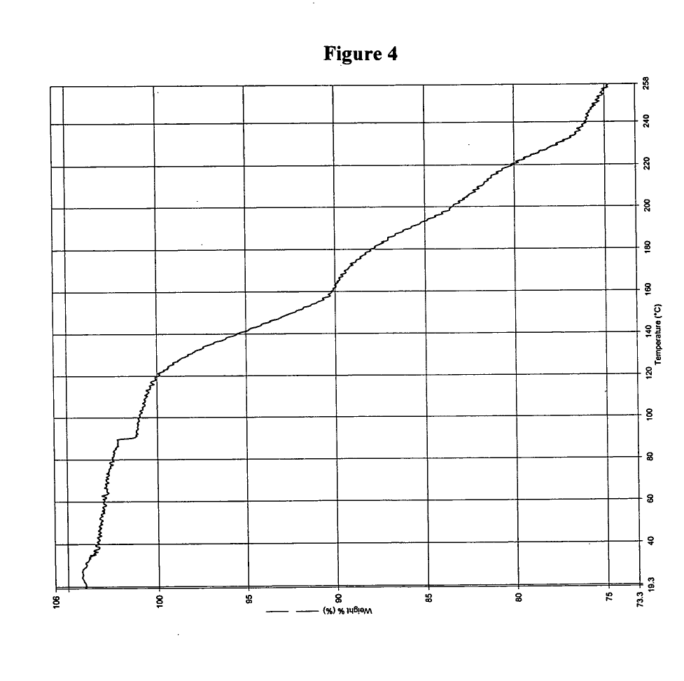

Still other characterizing data for crystalline rimonabant hydrochloride ethanolate according to the present invention as obtained by thermogravimetric analysis (TGA) is shown in FIG. 4, and it provides characteristic weight loss of about 8.5-9% from 120-160° C. region in its TGA thermogram, which is consistent with a composition of rimonabant hydrochloride and ethanol with a molar ratio of 1:1. Therefore, the data from TGA, DSC and IR clearly demonstrated that Form A of rimonabant hydrochloride is an ethanol solvate of rimonabant hydrochloride, and its formula is shown below:

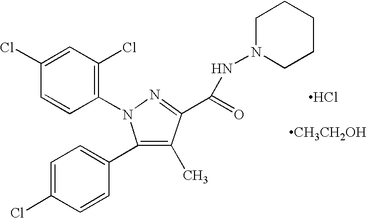

In one favored aspect, Form A of rimonabant hydrochloride provides X-ray powder diffraction (X-RPD) pattern substantially in accordance with FIG. 1 and Table 1.

In another favored aspect, Form A of rimonabant hydrochloride provides differential scanning calorimetry (DSC) substantially in accordance with FIG. 2.

In still another favored aspect, Form A of rimonabant hydrochloride provides infrared spectrum (IR) substantially in accordance with FIG. 3.

In a further favored aspect, Form A of rimonabant hydrochloride provides thermogravimetric analysis (TGA) thermogram substantially in accordance with FIG. 4.

The present invention encompasses Form A of rimonabant hydrochloride (crystalline rimonabant hydrochloride ethanolate) isolated in pure form or in a mixture as a solid composition when admixed with other materials, for example the known polymorph forms or solvate forms (i.e. Form B, C, D, E or amorphous form) of rimonabant hydrochloride or any other materials. The content of Form A of rimonabant hydrochloride A in a solid composition is over 50% (w/w), preferably 80% (w/w), more preferably over 95% (w/w), and most preferably over 98% (w/w).

Thus in one aspect there is provided Form A of rimonabant hydrochloride in isolated form.

In a further aspect there is provided Form A of rimonabant hydrochloride in pure form. The pure form means that polymorph Form A is over 95% (w/w), and preferably over 98% (w/w).

TABLE 1
X-ray diffraction pattern of Form A of rimonabant hydrochloride

| Peak No. | 2θ (deg) | FWHM | d-value | Intensity | I/Io |
|---|---|---|---|---|---|
| 1 | 10.20 | — | 8.6648 | 61 | 24 |
| 2 | 12.04 | 0.188 | 7.3445 | 166 | 65 |
| 3 | 12.76 | 0.188 | 6.9316 | 68 | 27 |
| 4 | 13.14 | 0.188 | 6.7320 | 198 | 78 |
| 5 | 13.62 | 0.259 | 6.4958 | 67 | 27 |
| 6 | 14.62 | 0.165 | 6.0537 | 163 | 64 |
| 7 | 16.76 | — | 5.2852 | 70 | 28 |
| 8 | 17.56 | 0.188 | 5.0462 | 86 | 34 |
| 9 | 17.88 | 0.188 | 4.9566 | 71 | 28 |
| 10 | 18.44 | 0.188 | 4.8073 | 122 | 48 |
| 11 | 19.18 | 0.165 | 4.6235 | 140 | 55 |
| 12 | 19.86 | 0.235 | 4.4667 | 177 | 70 |
| 13 | 20.90 | 0.306 | 4.24671 | 177 | 70 |
| 14 | 22.96 | 0.188 | 3.8701 | 67 | 27 |
| 15 | 23.96 | 0.188 | 3.7108 | 117 | 46 |
| 16 | 24.34 | 0.235 | 3.6537 | 128 | 50 |
| 17 | 24.84 | — | 3.5813 | 87 | 34 |
| 18 | 27.00 | 0.165 | 3.2995 | 93 | 37 |
| 19 | 27.46 | 0.282 | 3.2453 | 85 | 34 |
| 20 | 28.70 | 0.212 | 3.1078 | 61 | 25 |
| 21 | 29.28 | 0.165 | 3.0476 | 72 | 28 |
| 22 | 29.48 | 0.165 | 3.0273 | 56 | 22 |
| 23 | 30.18 | 0.165 | 2.9587 | 167 | 66 |

In yet a further aspect there is provided Form A of rimonabant hydrochloride in crystalline form.

In a preferable aspect, the particle size of Form A of rimonabant hydrochloride of the present invention has the median value of the volume mean diameter of the particles within the range of 0.01 μm-500 μm, preferably 5-300 μm, and most preferably 50-200 μm. Such particles are better in stability, good material flow characteristics and thus suitable for bulk preparation and formulation advantages.

The X-ray powder diffraction pattern, differential scanning calorimetry thermogram and infrared spectroscopy absorption bands of Form A of rimonabant hydrochloride is clearly different from those of other crystalline polymorph forms or solvates of rimonabant hydrochloride. As shown in FIG. 3, Form A of rimonabant hydrochloride has a characteristic infrared spectroscopy absorption band at 3254 cm⁻¹ due to the presence of —OH group in ethanol. The data from X-ray powder diffraction pattern, IR, TGA and DSC clearly demonstrated that Form A of rimonabant hydrochloride of the present invention is a new crystalline ethanol solvate form of rimonabant hydrochloride, and the molar ratio of rimonabant hydrochloride to ethanol is 1:1, and it is also termed as rimonabant hydrochloride ethanolate.

In another aspect, the present invention provides a process for preparing Form A of rimonabant hydrochloride, in which an appropriate alcohol solvent is used as crystallization or recrystallization media. The alcohol solvent of the present invention is ethanol or a mixture of ethanol with water or with any C₁-C₄ alcohols such as methanol, isopropyl alcohol, n-butyl alcohol. In the preferred embodiment of the present invention, the alcohol solvent is ethanol. In the most preferred embodiment of the present invention the alcohol solvent is absolute ethanol. The selected approach leads to Form A of rimonabant hydrochloride which contains one molecule of rimonabant hydrochloride and one molecule of ethanol (or the molar ratio is 1:1).

Specifically, the present invention also provides a process for preparation of Form A of rimonabant hydrochloride. A slurry of starting material, crude rimonabant hydrochloride or any polymorph forms of rimonabant hydrochloride, which can be obtained according to the procedure in Example 196 of U.S. Pat. No. 5,624,941 or any other procedures, admixed with ethanol is heated, suitably to a temperature in the range of from 35° C. to 90° C., such as 60° C. to 75° C., for example 70° C. until all solid materials are dissolved. The hot solution is allowed to cool down to ambient temperature, and the cooled solution is kept at −10° C. to 25° C. for crystallization, preferably at 0 to 10° C., and more preferably at 5° C. The Form A of rimonabant hydrochloride is recovered from the solvent by filtration at a temperature of 10° C. to 20° C. Optionally, the solution mixture is seeded with the Form A of rimonabant hydrochloride.

More specifically, the crude starting material, rimonabant hydrochloride can be dissolved in solvent at any temperature which provides a suitable rate of formation of the a clear supersaturated solution, generally at an elevated temperature for example at the reflux temperature of the solvent and thereafter crystallizing the required product at low temperature, preferably −10° C. to 25° C., more preferably 0° C. to 10° C. The Form A of rimonabant hydrochloride is recovered from the solvent by removing the solvent, e.g., by filtration at a temperature of 10° C. to 20° C. The obtained product is dried under vacuum oven or other driers, at a temperature of 25° C. to 65° C., preferably, 35° C. to 55° C., and most preferably, 40° C. to 45° C. The drying time is from 1 hour to 60 hours, preferably 5-20 hours or until the weight of the product is constant.

Form A of rimonabant hydrochloride as obtained above is characterized by powder x-ray diffraction pattern, substantially as shown in FIG. 1 and Table 1.

Form A of rimonabant hydrochloride as obtained above is characterized by differential scanning calorimetry (DSC), substantially as shown in FIG. 2.

Form A of rimonabant hydrochloride as obtained above is characterized by FT-Infrared spectrum (FT-IR), substantially as shown in FIG. 3.

Form A of rimonabant hydrochloride as obtained above is characterized by thermogravimetric analysis (TGA) thermogram, substantially as shown in FIG. 4.

Form A of rimonabant hydrochloride as obtained above is a crystalline ethanol solvate of rimonabant hydrochloride, containing about 8.5-9% ethanol by weight.

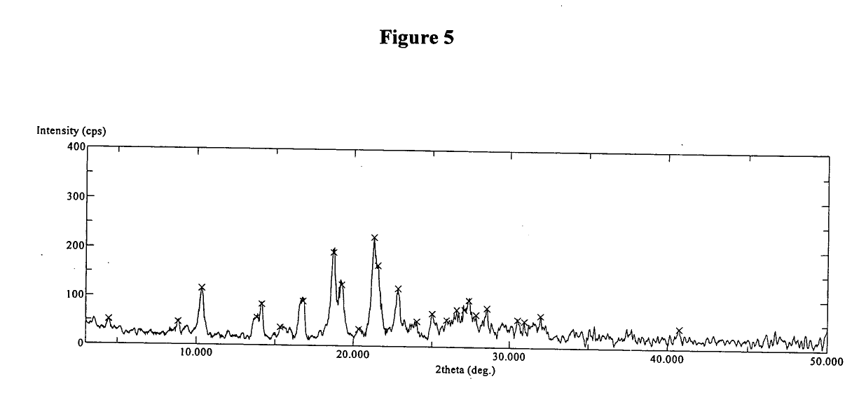

Accordingly, the present invention provides a novel and well-defined crystalline dichloromethane solvate form of rimonabant hydrochloride, also termed as crystalline rimonabant hydrochloride dichloromethanate with a molar ratio of 3:1, designed hereinafter as Form B of rimonabant hydrochloride, having an X-ray powder diffraction pattern, or substantially the same X-ray powder diffraction pattern, as shown in FIG. 5.

More particularly, Form B of rimonabant hydrochloride according to the present invention can be characterized as having an X-ray diffraction pattern with characteristic peaks (expressed in degrees 2θ, accuracy is +/−0.2 degree): 8.82, 10.32, 13.84, 14.14, 15.32, 16.67, 18.70, 19.24, 20.34, 21.26, 21.52, 22.82, 24.02, 25.00, 25.92, 26.58, 27.08, 27.38, 27.84, 28.54, 30.50, 30.94, 31.96 and 40.74.

Characterizing data for Form B of rimonabant hydrochloride according to the present invention as obtained by X-ray powder diffraction pattern is shown in FIG. 5 and Table 2.

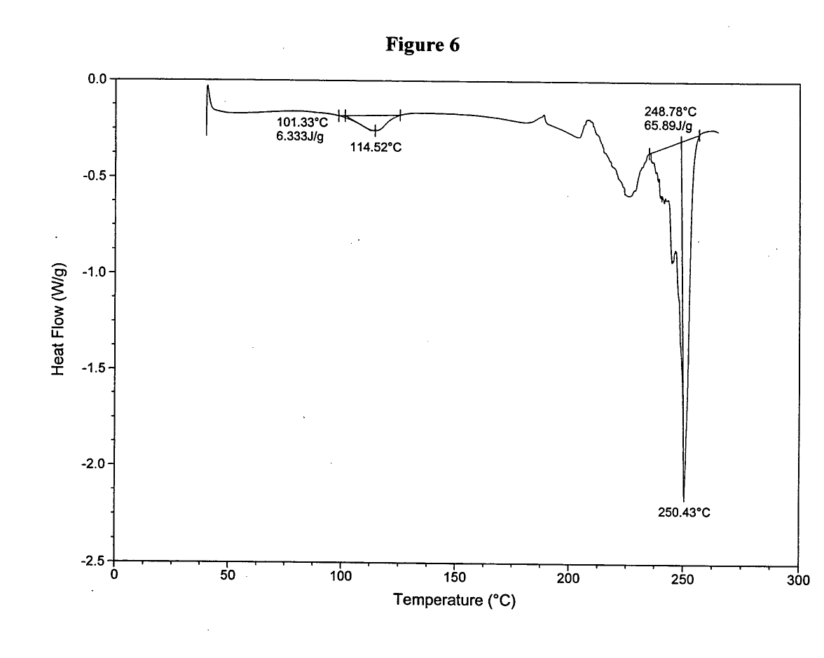

Further characterizing data for Form B of rimonabant hydrochloride according to the present invention as obtained by differential scanning calorimetry (DSC) is shown in FIG. 6, and it provides characteristic peaks at about 110° C. to 114° C. and about 250° C. to 254° C.

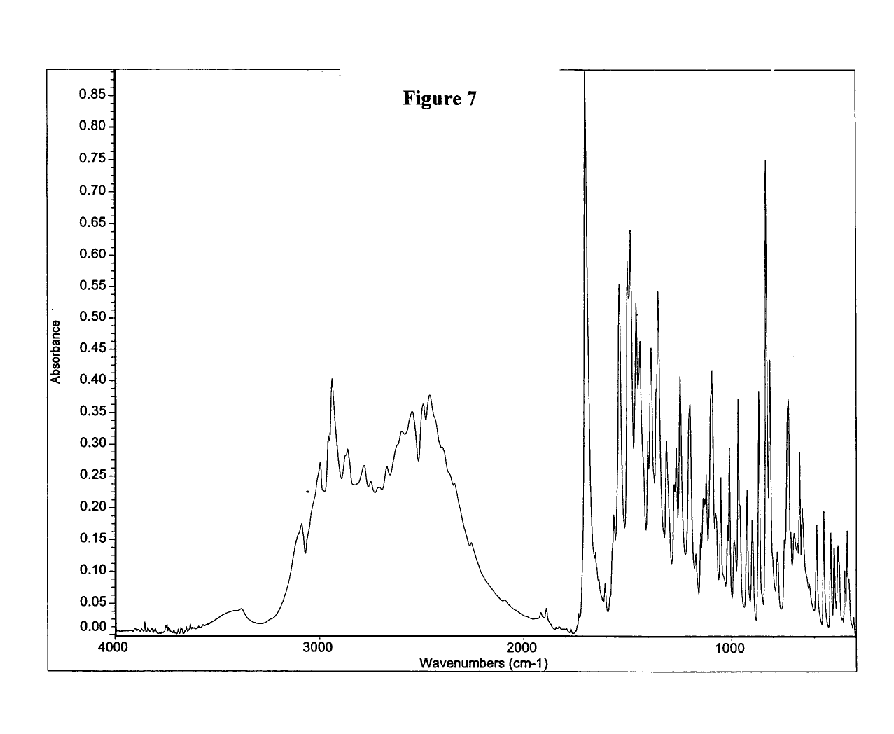

Still further characterizing data for Form B of rimonabant hydrochloride according to the present invention as obtained by infrared spectroscopy (KBr adsorption method) is shown in FIG. 7, and it provides characteristic absorbance peaks at 3087, 2997, 2938, 2862, 2549, 2464, 1700, 1565, 1538, 1497, 1483, 1456, 1438, 1402, 1385, 1351, 1312, 1265, 1246, 1200, 1122, 1096, 1053, 1011, 989, 969, 926, 902, 869, 834, 816, 781, 727, 699, 673, 661, 588, 554, 520, 504, 485 cm⁻¹.

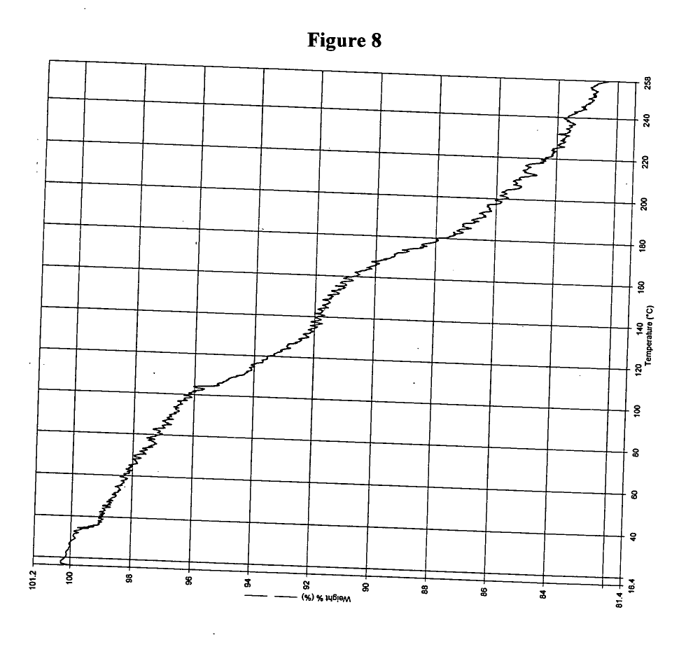

Still other characterizing data for polymorph Form B of crystalline rimonabant hydrochloride according to the present invention as obtained by thermogravimetric analysis (TGA) thermogram is shown in FIG. 8, and it provides characteristic weight loss of about 5% from 100° C. to 160° C., which is consistent with a composition of three molecules of rimonabant hydrochloride and one molecule of dichloromethane (or the molar ratio is 3:1).

In one favored aspect, Form B of rimonabant hydrochloride provides X-ray powder diffraction (X-RPD) pattern substantially in accordance with FIG. 5 and Table 2.

In another favored aspect, Form B of rimonabant hydrochloride provides differential scanning calorimetry (DSC) substantially in accordance with FIG. 6.

In still another favored aspect, Form B of rimonabant hydrochloride provides infrared spectrum (IR) substantially in accordance with FIG. 7.

In a further favored aspect, Form B of rimonabant hydrochloride provides thermogravimetric analysis (TGA) thermogram substantially in accordance with FIG. 8.

TABLE 2
X-ray diffraction pattern of Form B of rimonabant hydrochloride

| | | FWHM | | Intensity | |
|---|---|---|---|---|---|
| Peak No. | 2θ (deg) | (deg) | d-value | (counts) | I/I₀ |
| 1 | 4.460 | ***** | 19.7953 | 51 | 24 |
| 2 | 8.820 | ***** | 10.0172 | 47 | 22 |
| 3 | 10.320 | 0.259 | 8.5644 | 116 | 53 |
| 4 | 13.840 | 0.165 | 6.3930 | 57 | 26 |
| 5 | 14.140 | 0.212 | 6.2581 | 84 | 38 |
| 6 | 15.320 | ***** | 5.7786 | 37 | 17 |
| 7 | 16.760 | 0.494 | 5.2852 | 91 | 41 |
| 8 | 18.700 | 0.306 | 4.7411 | 192 | 86 |
| 9 | 19.240 | 0.329 | 4.6092 | 125 | 57 |
| 10 | 20.340 | ***** | 4.3623 | 34 | 16 |
| 11 | 21.260 | 0.235 | 4.1756 | 223 | 100 |
| 12 | 21.520 | 0.188 | 4.1257 | 165 | 75 |
| 13 | 22.820 | 0.329 | 3.8936 | 119 | 54 |
| 14 | 24.020 | ***** | 3.7017 | 52 | 24 |
| 15 | 25.000 | 0.212 | 3.5588 | 68 | 31 |
| 16 | 25.920 | 0.165 | 3.4345 | 55 | 25 |
| 17 | 26.580 | 0.165 | 3.3507 | 77 | 35 |
| 18 | 27.080 | 0.165 | 3.2899 | 77 | 35 |
| 19 | 27.380 | 0.212 | 3.2546 | 96 | 44 |
| 20 | 27.840 | 0.235 | 3.2018 | 67 | 30 |
| 21 | 28.540 | 0.235 | 3.1249 | 80 | 37 |
| 22 | 30.500 | 0.259 | 2.9284 | 57 | 26 |
| 23 | 30.940 | 0.165 | 2.8877 | 54 | 25 |
| 24 | 31.960 | 0.165 | 2.7979 | 66 | 30 |
| 25 | 40.740 | ***** | 2.2129 | 42 | 19 |

The present invention encompasses Form B of rimonabant hydrochloride isolated in pure form or in a mixture as a solid composition when admixed with other materials, for example the known polymorph forms or solvate forms (i.e. Form A, C, D, E or amorphous form) of rimonabant hydrochloride or any other material. The content of Form B of rimonabant hydrochloride in a solid composition is over 50% (w/w), preferably 80% (w/w), more preferably over 95% (w/w), and most preferably over 98% (w/w).

Thus in one aspect there is provided Form B of rimonabant hydrochloride in isolated form.

In a further aspect there is provided Form B of rimonabant hydrochloride in pure form. The pure form means that Form B of rimonabant hydrochloride is over 95% (w/w), and preferably over 98% (w/w).

In yet a further aspect there is provided Form B of rimonabant hydrochloride in crystalline form.

In a preferable aspect, the particle size of Form B of rimonabant hydrochloride of the present invention has the median value of the volume mean diameter of the particles within the range of 0.01 μm-500 μm, preferably 5-300 μm, and most preferably 50-200 μm. Such particles are better in stability, good material flow characteristics and thus suitable for bulk preparation and formulation advantages.

The X-ray powder diffraction pattern, differential scanning calorimetry (DSC), infrared spectrum (IR) of Form B of rimonabant hydrochloride is clearly different from those of other polymorph forms. Therefore, Form B of rimonabant hydrochloride of the present invention is a new crystalline solvate form of rimonabant hydrochloride. The data from DSC and TGA data clearly demonstrated that this novel crystalline form of rimonab ant hydrochloride is a dichloromethane solvate of crystalline rimonabant hydrochloride (also termed as rimonabant hydrochloride dichloromethanate), and the molar ratio of rimonabant hydrochloride to dichloromethane in the form is 3:1, as shown by formula below:

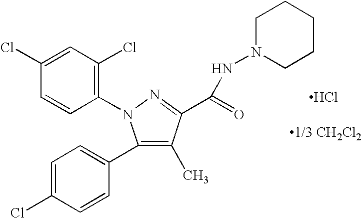

In another aspect, the present invention provides a process for preparing Form B of rimonabant hydrochloride, in which an appropriate dichloromethane solvent is used as crystallization or recrystallization media. The dichloromethane solvent of the present invention can also be or a mixture of dichloromethane with any other organic solvents. In the preferred embodiment of the present invention, the dichloromethane solvent is pure dichloromethane. The selected approach leads to Form B of rimonabant hydrochloride that contains three molecules of rimonabant hydrochloride and one molecule of dichloromethane.

Specifically, the present invention also provides a process for preparation of Form B of rimonabant hydrochloride. A slurry of starting material, crude rimonabant hydrochloride or any polymorph forms of crystalline rimonabant hydrochloride, which can be obtained according to the procedure in Example 196 of U.S. Pat. No. 5,624,941 or any other procedures, admixed with dichloromethane is heated, suitably to a temperature in the range of from 35° C. to 70° C., such as 50° C. to 60° C., for example 55° C. until all solid materials are dissolved. The hot solution is allowed to cool down to ambient temperature, and the cooled solution is kept at −18° C.-25° C. for crystallization, preferably at 0° C. to 10° C., and more preferably at 5° C. Form B of rimonabant hydrochloride is recovered from the solvent by filtration at a temperature of 10° C. to 20° C. Optionally, the solution mixture is seeded with Form B of rimonabant hydrochloride.

More specifically, the crude starting material, rimonabant hydrochloride, can be dissolved in dichloromethane solvent at any temperature which provides a suitable rate of formation of the a clear supersaturated solution, generally at an elevated temperature for example at the reflux temperature of the solvent and thereafter crystallizing the required product at low temperature, preferably −10° C. to 25° C., more preferably 0° C. to 10° C. Form B of rimonabant hydrochloride is recovered from the solvent by removing the solvent, e.g., by filtration at a temperature of 10° C. to 20° C. The obtained product is dried under vacuum oven or other driers, at a temperature of 25° C. to 65° C., preferably, 35° C. to 55° C., and most preferably, 38° C. to 45° C. The drying time is from 1 hour to 60 hours, preferably 5-20 hours or until the weight of the product is constant.

Form B of rimonabant hydrochloride as obtained above is characterized by powder X-ray diffraction pattern, substantially as shown in FIG. 5 and Table 2.

Form B of rimonabant hydrochloride as obtained above is characterized by differential scanning calorimetry (DSC), substantially as shown in FIG. 6.

Form B of rimonabant hydrochloride as obtained above is characterized by FT-Infrared spectrum (FT-IR), substantially as shown in FIG. 7.

Form B of rimonabant hydrochloride as obtained above is characterized by thermogravimetric analysis (TGA) thermogram, substantially as shown in FIG. 8.

Form B of rimonabant hydrochloride as obtained above is a crystalline dichloromethane solvate of rimonabant hydrochloride, containing about 5% dichloromethane by weight, or with a molar ratio of rimonabant hydrochloride to dichloromethane being 3:1.

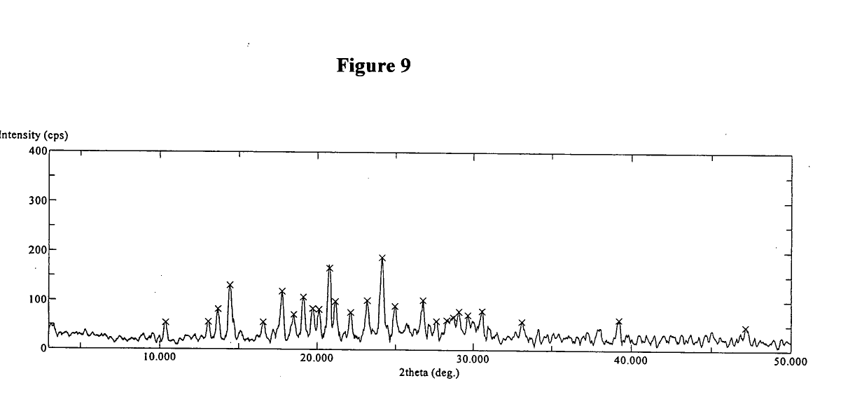

Accordingly, the present invention further provides a novel and well-defined crystalline hemi-isopropanol solvate of rimonabant hydrochloride, also termed as rimonabant hydrochloride hemi-isopropanolate (hereinafter designed as Form C of rimonabant hydrochloride), having an X-ray powder diffraction pattern, or substantially the same X-ray powder diffraction pattern, as shown in FIG. 9.

More particularly, Form C of rimonabant hydrochloride according to the present invention can be characterized as having an X-ray diffraction pattern with characteristic peaks (expressed in degrees 2θ, accuracy is +/−0.2 degree): 10.38, 13.06, 13.68, 14.44, 16.54, 17.76, 18.52, 19.12, 19.72, 20.12, 20.78, 21.16, 22.14, 23.18, 24.12, 24.96, 26.78, 27.64, 28.30, 28.72, 29.08, 29.66, 30.56, 33.10 and 39.22.

Characterizing data for Form C of rimonabant hydrochloride according to the present invention as obtained by X-ray powder diffraction pattern is shown in FIG. 9 and Table 3.

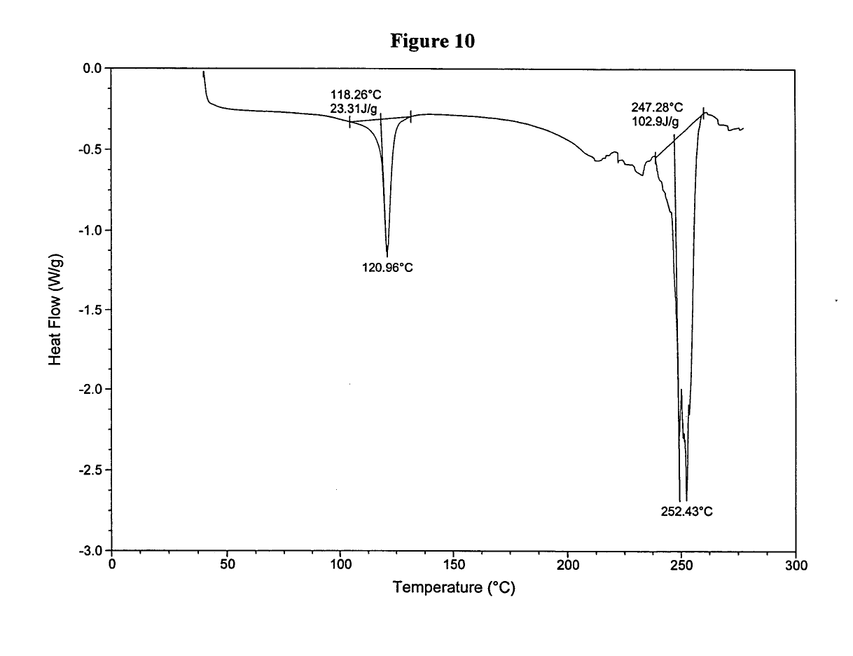

Further characterizing data for Form C of rimonabant hydrochloride according to the present invention as obtained by differential scanning calorimetry (DSC) is shown in FIG. 10, and it provides a characteristic peak at about 116-122° C., another characteristic peak at about 250-254° C.

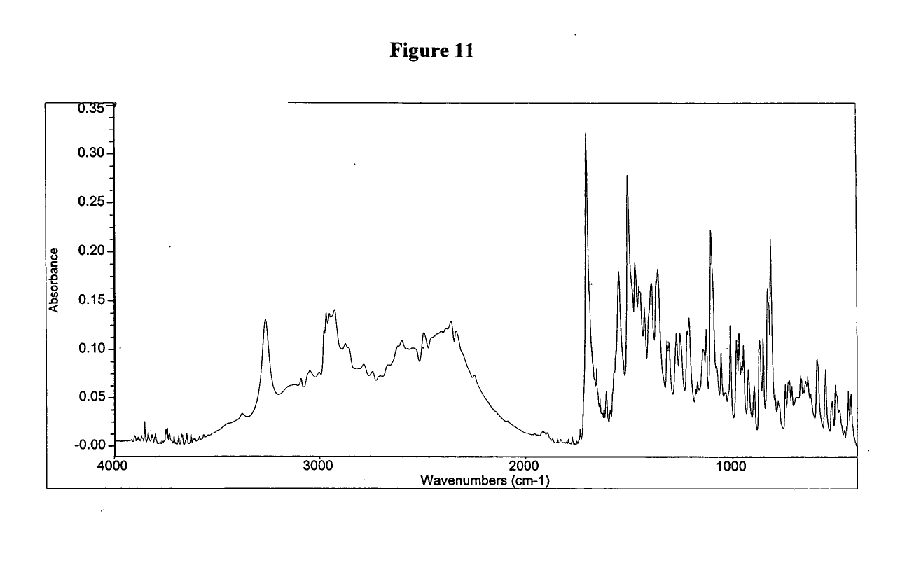

Still further characterizing data for Form C of rimonabant hydrochloride according to the present invention as obtained by infrared spectroscopy (by KBr adsorption method) is shown in FIG. 11, and it provides characteristic absorbance peaks at 3262, 2967, 2927, 2602, 2496, 2364, 1699, 1653, 1606, 1541, 1496, 1463, 1444, 1419, 1385, 1354, 1310, 1267, 1250, 1207, 1140, 1124, 1101, 1055, 1010, 982, 969, 949, 925, 897, 871, 854, 831, 815, 781, 747, 728, 715, 673, 638, 593, 552, 520, 504, 441 and 428 cm⁻¹. The adsorption band 3262 cm⁻¹ is from the —OH group of isopropanol solvent in Form C of rimonabant hydrochloride.

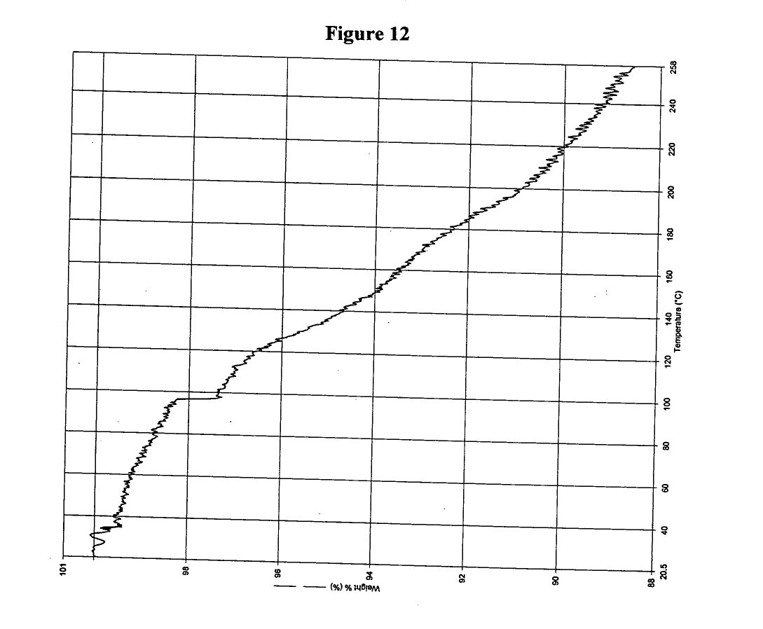

Still other characterizing data for Form C of rimonabant hydrochloride according to the present invention as obtained by thermogravimetric analysis (TGA) thermogram is shown in FIG. 12, and it provides characteristic weight loss of about 5% from 102° C. to 165° C., which is consistent with a composition of rimonabant hydrochloride and isopropyl alcohol with a molar ratio of 1:0.5.

In one favored aspect, Form C of rimonabant hydrochloride provides X-ray powder diffraction (X-RPD) pattern substantially in accordance with FIG. 9 and Table 3.

In another favored aspect, Form C of rimonabant hydrochloride provides differential scanning calorimetry (DSC) thermogram substantially in accordance with FIG. 10.

In still another favored aspect, Form C of rimonabant hydrochloride provides infrared spectrum (IR) substantially in accordance with FIG. 11.

In a further favored aspect, Form C of rimonabant hydrochloride provides thermogravimetric analysis (TGA) thermogram substantially in accordance with FIG. 12.

TABLE 3
X-ray diffraction pattern of Form C of rimonabant hydrochloride

| | | FWHM | | Intensity | |
|---|---|---|---|---|---|
| Peak No. | 2θ (deg) | (deg) | d-value | (counts) | I/I₀ |
| 1 | 10.38 | | 8.5150 | 55 | 29 |
| 2 | 13.060 | 0.259 | 6.7731 | 56 | 30 |
| 3 | 13.68 | 0.259 | 6.4675 | 82 | 44 |
| 4 | 14.14 | 0.259 | 6.41287 | 131 | 70 |
| 5 | 16.54 | | 5.3550 | 56 | 30 |
| 6 | 17.76 | 0.259 | 4.9898 | 119 | 63 |
| 7 | 18.52 | 0.235 | 4.7868 | 72 | 38 |
| 8 | 19.12 | 0.259 | 4.6378 | 107 | 59 |
| 9 | 19.72 | 0.259 | 4.498 1 | 84 | 45 |
| 10 | 20.12 | 0.235 | 4.4095 | 82 | 44 |
| 11 | 20.78 | 0.259 | 4.2710 | 166 | 89 |
| 12 | 20.16 | 0.259 | 4.1951 | 98 | 53 |
| 13 | 22.14 | 0.235 | 4.0116 | 77 | 41 |
| 14 | 23.18 | 0.259 | 3.8339 | 101 | 54 |
| 15 | 24.12 | 0.306 | 3.6866 | 189 | 100 |
| 16 | 24.96 | 0.259 | 3.5644 | 29 | 48 |
| 17 | 26.78 | 0.366 | 3.3261 | 101 | 54 |
| 18 | 27.64 | 0.282 | 3.2245 | 59 | 32 |
| 19 | 28.30 | 0.235 | 3.1508 | 61 | 33 |
| 20 | 28.72 | 0.165 | 3.1057 | 67 | 36 |
| 21 | 29.08 | 0.235 | 3.0681 | 79 | 42 |
| 22 | 29.66 | 0.235 | 3.0094 | 72 | 39 |
| 23 | 30.56 | 0.259 | 2.9228 | 80 | 43 |
| 24 | 33.10 | 0.235 | 2.7041 | 58 | 31 |
| 25 | 39.22 | 0.306 | 2.2950 | 63 | 34 |

The present invention encompasses Form C of rimonabant hydrochloride isolated in pure form or in a mixture as a solid composition when admixed with other materials, for example the known polymorph forms or solvates (i.e. Form A, B, D, E or amorphous form) of rimonabant hydrochloride or any other material. The content of polymorph Form C in a solid composition is over 50% (w/w), preferably 80% (w/w), more preferably over 95% (w/w), and most preferably over 98% (w/w).

Thus in one aspect there is provided Form C of rimonabant hydrochloride in isolated form.

In a further aspect there is provided Form C of rimonabant hydrochloride in pure form. The pure form means that Form C of rimonabant hydrochloride is over 95% (w/w), and preferably over 98% (w/w).

In yet a further aspect there is provided Form C of rimonabant hydrochloride in crystalline form.

In a preferable aspect, the particle size of Form C of the present invention has the median value of the volume mean diameter of the particles within the range of 0.01 μm-500 μm, preferably 5-300 μm, and most preferably 50-200 μm. Such particles are better in stability, good material flow characteristics and thus suitable for bulk preparation and formulation advantages.

The X-ray powder diffraction pattern, differential scanning calorimetry (DSC), infrared spectrum (IR) of Form C of rimonabant hydrochloride is clearly different from those of other polymorphic forms or solvates. Therefore, Form C of rimonabant hydrochloride of the present invention is a new crystalline solvate form of rimonabant hydrochloride. From DSC and TGA data, it is clear that Form C of rimonabant hydrochloride is a novel crystalline hemi-isopropanol solvate of rimonabant hydrochloride (also termed as rimonabant hydrochloride hemi-isopropanolate), and the molar ratio of rimonabant hydrochloride to isopropyl alcohol is 1:0.5, as shown by formula below:

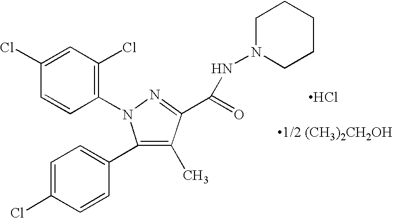

In another aspect, the present invention provides a process for preparing Form C of rimonabant hydrochloride, in which an appropriate organic solvent is used as crystallization or recrystallization media. The organic solvent of the present invention can be isopropyl alcohol or a mixture of isopropyl alcohol with any other organic solvents such as tetrahydrofuran (THF) or ethyl acetate. In the preferred embodiment of the present invention, the organic solvent is pure isopropyl alcohol. In the more preferred embodiment of the present invention, the organic solvent is a mixture of isopropyl alcohol, ethyl acetate and tetrahydrofuran with a ratio of 1:0.1-2:0.1-2 (v/v/v). In the most preferred embodiment of the present invention, the organic solvent is a mixture of isopropyl alcohol, ethyl acetate and tetrahydrofuran with a ratio of 1:0.5:0.5 (v/v/v). The selected approach leads to the crystalline Form C of rimonabant hydrochloride which contains rimonabant hydrochloride and isopropyl alcohol in a molar ratio of 1:0.5.

Specifically, the present invention also provides a process for preparation of Form C of rimonabant hydrochloride. A slurry of starting material, crude rimonabant hydrochloride or any polymorph forms of crystalline rimonabant hydrochloride, which can be obtained according to the procedure in Example 196 of U.S. Pat. No. 5,624,941 or any other procedures, admixed with isopropyl alcohol, ethyl acetate and tetrahydrofuran (e.g., 1:0.5:0.5, v/v/v) is heated, suitably to a temperature in the range of from 35° C. to 90° C., such as 50° C. to 80° C., for example 75° C. until all solid materials are dissolved. The hot solution is allowed to cool down to ambient temperature, and the cooled solution is kept at −10° C.-25° C. for crystallization, preferably at 0° C. to 10° C., and more preferably at 5° C. Form C of rimonabant hydrochloride is recovered from the solvent by filtration at a temperature of 10° C. to 20° C. Optionally, the solution mixture is seeded with Form C of rimonabant hydrochloride.

More specifically, the crude starting material, rimonabant hydrochloride, can be dissolved in isopropyl alcohol, ethyl acetate and tetrahydrofuran (e.g., 1:0.5:0.5, v/v/v) solvent at any temperature which provides a suitable rate of formation of the a clear supersaturated solution, generally at an elevated temperature for example at the reflux temperature of the solvent and thereafter crystallizing the required product at low temperature, preferably −10° C. to 25° C., more preferably 0° C. to 10° C. Form C of rimonabant hydrochloride is recovered from the solvent by removing the solvent, e.g., by filtration. The obtained product is dried under vacuum oven or other driers, at a temperature of 25° C. to 65° C., preferably, 35° C. to 55° C., and most preferably, 40° C. to 45° C. The drying time is from 1 hour to 60 hours, preferably 5-20 hours or until the weight of the product is constant.

Form C of rimonabant hydrochloride as obtained above is characterized by powder X-ray diffraction pattern, substantially as shown in FIG. 9 and Table 3.

Form C of rimonabant hydrochloride as obtained above is characterized by differential scanning calorimetry (DSC) thermogram, substantially as shown in FIG. 10.

Form C of rimonabant hydrochloride as obtained above is characterized by FT-Infrared spectrum (FT-IR), substantially as shown in FIG. 11.

Form C of rimonabant hydrochloride as obtained above is characterized by thermogravimetric analysis (TGA) thermogram, substantially as shown in FIG. 12.

Form C of rimonabant hydrochloride as obtained above is a crystalline isopropyl alcohol solvate of rimonabant hydrochloride, containing about 5% isopropyl alcohol by weight.

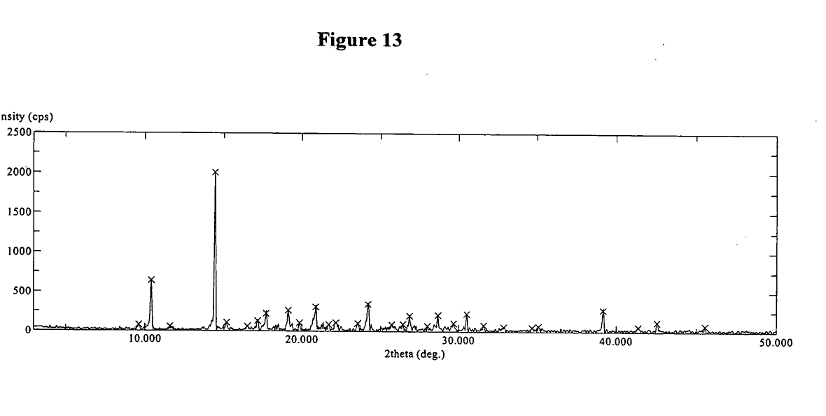

Accordingly, the present invention still provides a novel and well-defined crystalline polymorph of rimonabant hydrochloride anhydrous (hereinafter designed as Form D of rimonabant hydrochloride), having an X-ray powder diffraction pattern, or substantially the same X-ray powder diffraction pattern, as shown in FIG. 13.

More particularly, Form D of rimonabant hydrochloride according to the present invention can be characterized as having an X-ray powder diffraction pattern with characteristic peaks (expressed in degrees 2θ, accuracy is +/−0.2 degree): 9.60, 10.40, 11.58, 14.44, 15.18, 16.50, 17.16, 17.70, 19.12, 19.86, 20.88, 21.70, 22.16, 23.54, 24.18, 25.70, 26.44, 26.86, 28.00, 28.68, 29.68, 30.52, 31.60, 32.88, 34.68, 35.10, 39.16, 41.38 and 42.56.

Characterizing data for Form D of rimonabant hydrochloride according to the present invention as obtained by X-ray powder diffraction pattern is shown in FIG. 13 and Table 4.

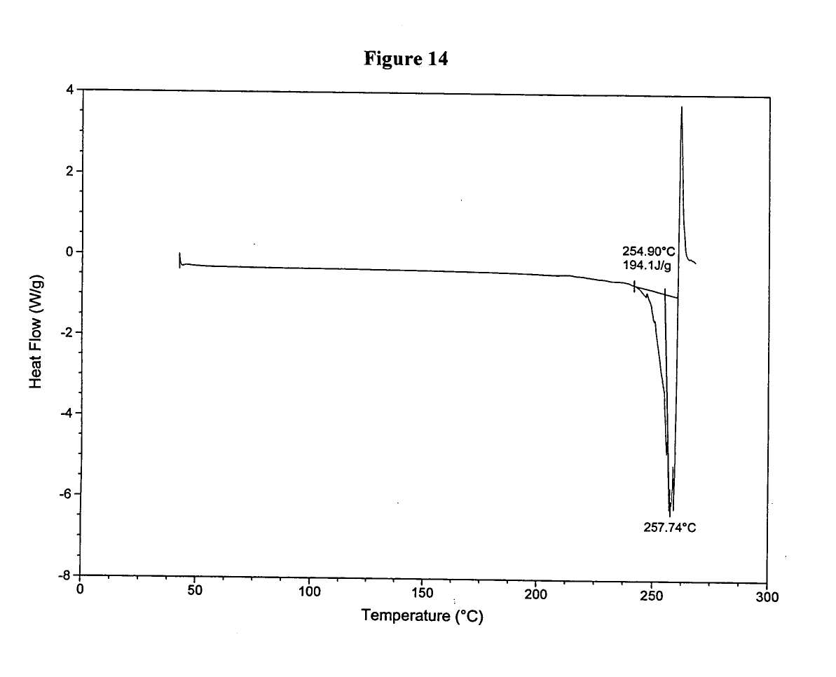

Further characterizing data for Form D of rimonabant hydrochloride according to the present invention as obtained by differential scanning calorimetry (DSC) is shown in FIG. 14, and it provides a characteristic peak at about 256-258° C.

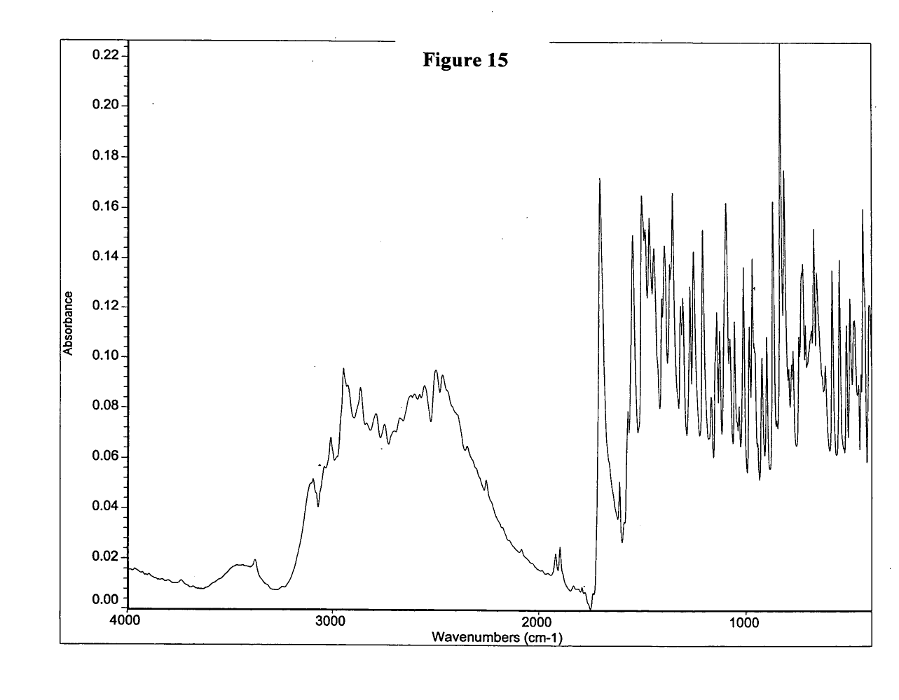

Still further characterizing data for Form D of rimonabant hydrochloride according to the present invention as obtained by infrared spectroscopy (by KBr adsorption method) is shown in FIG. 15, and it provides characteristic absorbance bands at 3095, 3009, 2946, 2864, 2552, 2496, 1700, 1566, 1541, 1498, 1462, 1440, 1402, 1389, 1348, 1311, 1300, 1267, 1250, 1206, 1167, 1140, 1126, 1094, 1077, 1054, 1032, 1011, 984, 969, 922, 900, 868, 833, 815, 773, 746, 726, 714, 673, 661, 619, 587, 552, 519, 503, 481 and 440 cm⁻¹.

Still other characterizing data for Form D of rimonabant hydrochloride according to the present invention as obtained by thermogravimetric analysis (TGA) is shown in FIG. 15, and it provides characteristic TGA thermogram of anhydrous (none solvate) rimonabant hydrochloride. The loss of weight at 160-260° C. range is mainly due to the decomposition and sublime of crystalline rimonabant hydrochloride at high temperature.

In one favored aspect, Form D of rimonabant hydrochloride provides X-ray powder diffraction (X-RPD) pattern substantially in accordance with FIG. 13 and Table 4.

In another favored aspect, Form D of rimonabant hydrochloride provides differential scanning calorimetry (DSC) thermogram substantially in accordance with FIG. 14.

In still another favored aspect, Form D of rimonabant hydrochloride provides infrared spectrum (IR) substantially in accordance with FIG. 15.

TABLE 4
X-ray diffraction pattern of Form D of rimonabant hydrochloride

| | | FWHM | | Intensity | |
|---|---|---|---|---|---|
| Peak No. | 2θ (deg) | (deg) | d-value | (counts) | I/I₀ |
| 1 | 9.6 | 0.212 | 9.2050 | 80 | 4 |
| 2 | 10.40 | 0.165 | 8.4987 | 647 | 33 |
| 3 | 11.58 | | 7.6352 | 57 | 3 |
| 4 | 14.44 | 0.165 | 6.1287 | 2005 | 100 |
| 5 | 15.18 | 0.165 | 5.8316 | 109 | 6 |
| 6 | 16.50 | 0.165 | 5.3679 | 62 | 4 |
| 7 | 17.16 | 0.165 | 5.1629 | 131 | 7 |
| 8 | 17.70 | 0.188 | 5.0066 | 226 | 12 |
| 9 | 19.12 | 0.188 | 4.6378 | 266 | 12 |
| 10 | 19.86 | 0.165 | 4.4667 | 106 | 6 |
| 11 | 20.88 | 0.165 | 4.2507 | 312 | 16 |
| 12 | 21.70 | 0.165 | 4.0919 | 89 | 5 |
| 13 | 22.16 | 0.259 | 4.0080 | 111 | 6 |
| 14 | 23.54 | 0.165 | 3.7761 | 104 | 6 |
| 15 | 24.18 | 0.188 | 3.6775 | 351 | 18 |
| 16 | 25.70 | 0.188 | 3.4634 | 89 | 5 |
| 17 | 26.44 | 0.165 | 3.3681 | 93 | 5 |
| 18 | 26.86 | 0.188 | 3.3164 | 200 | 10 |
| 19 | 28.00 | 0.188 | 3.1839 | 74 | 4 |
| 20 | 28.68 | 0.165 | 3.1099 | 211 | 11 |
| 21 | 29.68 | 0.329 | 3.0074 | 108 | 6 |
| 22 | 30.52 | 0.165 | 2.9265 | 227 | 12 |
| 23 | 31.60 | 0.165 | 2.8289 | 85 | 5 |
| 24 | 32.88 | 0.165 | 2.7216 | 62 | 4 |
| 25 | 34.68 | 0.165 | 2.5844 | 57 | 3 |
| 26 | 35.10 | | 2.5544 | 72 | 4 |
| 27 | 39.16 | 0.165 | 2.2984 | 279 | 14 |
| 28 | 41.38 | 0.212 | 2.1801 | 64 | 4 |
| 29 | 42.56 | 0.165 | 2.1223 | 123 | 7 |
| 30 | 45.56 | 0.165 | 1.9893 | 80 | 4 |

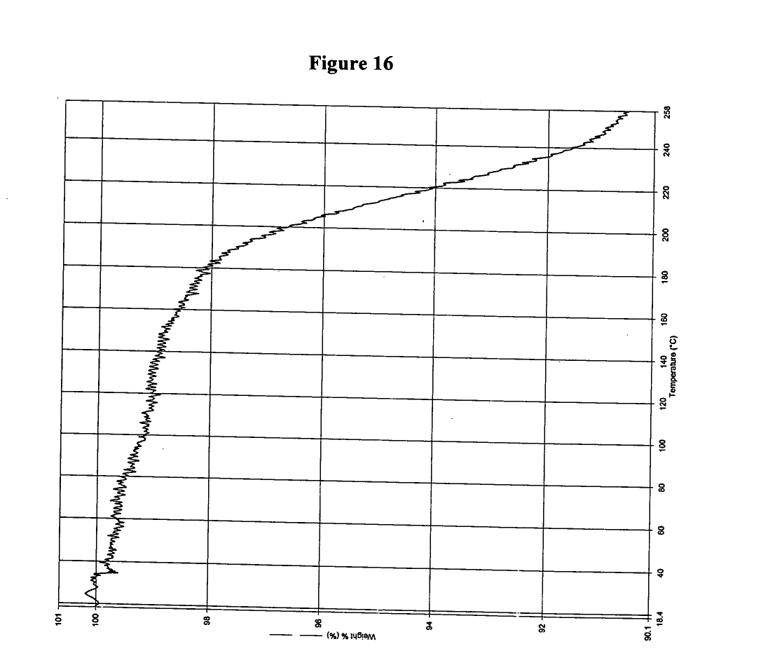

In a further favored aspect, Form D of rimonabant hydrochloride provides thermogravimetric analysis (TGA) thermogram substantially in accordance with FIG. 16.

The present invention encompasses Form D of rimonabant hydrochloride isolated in pure form or in a mixture as a solid composition when admixed with other materials, for example the known forms or solvates (i.e. Form A, B, C, E or amorphous form) of rimonabant hydrochloride or any other material. The content of Form D of rimonabant hydrochloride in a solid composition is over 50% (w/w), preferably 80% (w/w), more preferably over 95% (w/w), and most preferably over 98% (w/w).

Thus in one aspect there is provided Form D of rimonabant hydrochloride in isolated form.

In a further aspect there is provided Form D of rimonabant hydrochloride in pure form. The pure form means that Form D of rimonabant hydrochloride is over 95% (w/w), and preferably over 98% (w/w).

In yet a further aspect there is provided Form D of rimonabant hydrochloride in crystalline form.

In a preferable aspect, the particle size of Form D of rimonabant hydrochloride of the present invention has the median value of the volume mean diameter of the particles within the range of 0.01 μm-500 μm, preferably 5-300 μm, and most preferably 50-200 μm. Such particles are better in stability, good material flow characteristics and thus suitable for bulk preparation and formulation advantages.

The X-ray powder diffraction pattern, differential scanning calorimetry (DSC) thermogram, infrared spectrum (IR) of Form D of rimonabant hydrochloride is clearly different from those of other polymorph forms. Therefore, Form D of rimonabant hydrochloride of the present invention is a new crystalline polymorphic form of rimonabant hydrochloride anhydrous.

In another aspect, the present invention provides a process for preparing Form D of rimonabant hydrochloride, in which an appropriate organic solvent is used as crystallization or recrystallization media. The organic solvent of the present invention can be methanol, acetonitrile or a mixture of methanol and acetonitrile in any ratio. In the preferred embodiment of the present invention, the organic solvent is pure methanol or acetonitrile. In the more preferred embodiment of the present invention, the organic solvent is a mixture of acetonitrile and methanol with a ratio of 1:0.1-10 (v/v). In the most preferred embodiment of the present invention, the organic solvent is a mixture of acetonitrile and methanol with a ratio of 3:1 (v/v). The selected approach leads to Form D of rimonabant hydrochloride.

Specifically, the present invention also provides a process for preparation of Form D of rimonabant hydrochloride. A slurry of starting material, crude rimonabant hydrochloride or any polymorphic forms of crystalline rimonabant hydrochloride or its solvates or hydrates, which can be obtained according to the procedure in Example 196 of U.S. Pat. No. 5,624,941 or any other procedures, admixed with acetonitrile or methanol or a mixture of acetonitrile and methanol is heated, suitably to a temperature in the range of from 35° C. to 85° C., such as 50° C. to 70° C., for example 65° C. until all solid materials are dissolved. The hot solution is allowed to cool down to ambient temperature, and the cooled solution is kept at −10° C. to 25° C. for crystallization, preferably at 0° C. to 10° C., and more preferably at 5° C. Form D of rimonabant hydrochloride is recovered from the solvent by filtration at a temperature of 10° C. to 20° C. Optionally, the solution mixture is seeded with Form D of rimonabant hydrochloride.

More specifically, the crude starting material, rimonabant hydrochloride, can be dissolved in methanol, acetonitrile or a mixture of methanol and acetonitrile solvent at any temperature which provides a suitable rate of formation of the a clear supersaturated solution, generally at an elevated temperature for example at the reflux temperature of the solvent and thereafter crystallizing the required product at low temperature, preferably −18° C. to 25° C., more preferably 0° C. to 10° C. Form D of rimonabant hydrochloride is recovered from the solvent by removing the solvent, e.g., by filtration at a temperature of 10° C. to 20° C. The obtained product is dried under vacuum oven or other driers, at a temperature of 25° C. to 65° C., preferably, 35° C. to 55° C., and most preferably, 40° C. to 45° C. The drying time is from 1 hour to 60 hours, preferably 5-20 hours or until the weight of the product is constant.

Form D of rimonabant hydrochloride as obtained above is characterized by powder X-ray powder diffraction pattern, substantially as shown in FIG. 13 and Table 4.

Form D of rimonabant hydrochloride as obtained above is characterized by differential scanning calorimetry (DSC) thermogram, substantially as shown in FIG. 14.

Form D of rimonabant hydrochloride as obtained above is characterized by FT-Infrared spectrum (FT-IR), substantially as shown in FIG. 15.

Form D of rimonabant hydrochloride as obtained above is characterized by thermogravimetric analysis (TGA) thermogram, substantially as shown in FIG. 16.

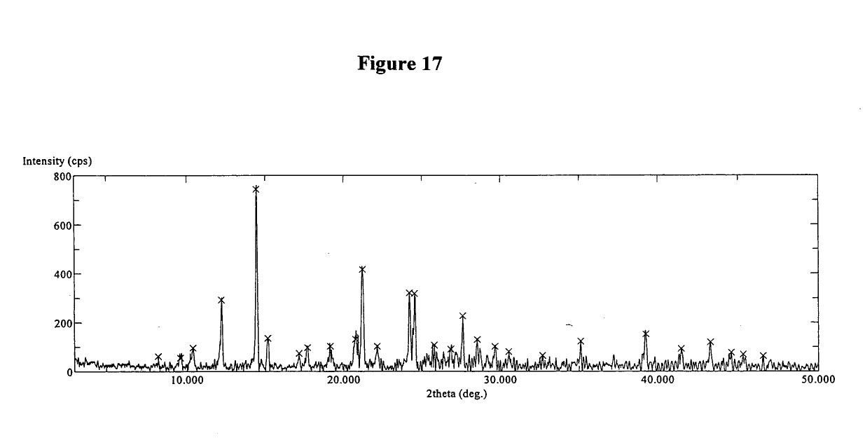

Accordingly, the present invention further provides a novel and well-defined crystalline polymorphic form of rimonabant hydrochloride anhydrous (hereinafter designed as Form E of rimonabant hydrochloride), having an X-ray powder diffraction pattern, or substantially the same X-ray powder diffraction pattern, as shown in FIG. 17.

More particularly, Form E of rimonabant hydrochloride according to the present invention can be characterized as having an X-ray powder diffraction pattern with characteristic peaks (expressed in degrees 2θ, accuracy is +/−0.2 degree): 9.58, 10.38, 12.18, 14.44, 15.14, 17.14, 17.68, 19.14, 20.76, 21.20, 22.14, 24.20, 24.52, 25.76, 26.82, 27.60, 28.52, 29.64, 30.52, 32.66, 35.10, 39.24, 41.46 and 43.28.

Characterizing data for Form E of rimonabant hydrochloride according to the present invention as obtained by X-ray powder diffraction pattern is shown in FIG. 17 and Table 5.

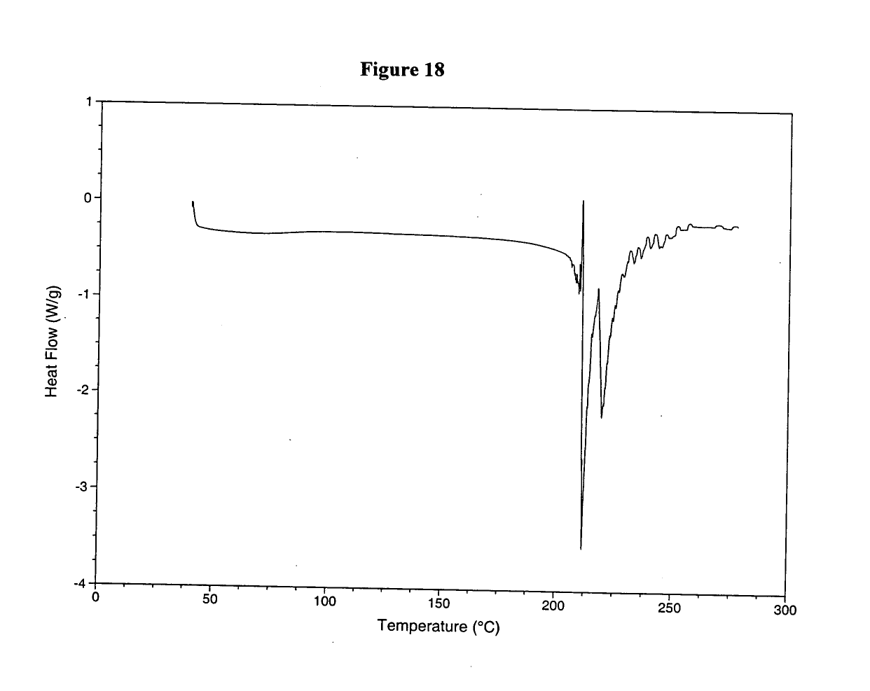

Further characterizing data for Form E of rimonabant hydrochloride according to the present invention as obtained by differential scanning calorimetry (DSC) is shown in FIG. 18, and it provides a characteristic peak or melting range at about 212-220° C.

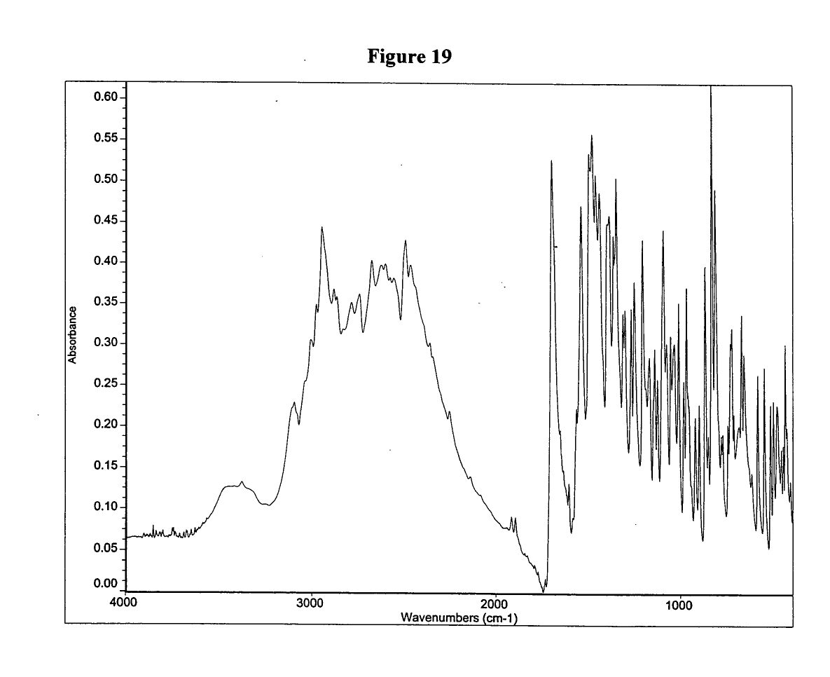

Still further characterizing data for Form E of rimonabant hydrochloride according to the present invention as obtained by infrared spectroscopy (by KBr adsorption method) is shown in FIG. 19, and it provides characteristic absorbance bands at 3095, 2946, 2742, 2675, 2601, 2492, 1700, 1606, 1540, 1480, 1461, 1440, 1387, 1363, 1348, 1311, 1300, 1267, 1250, 1206, 1169, 1140, 1126, 1094, 1077, 1054, 1034, 1011, 983, 969, 922, 900, 869, 851, 833, 814, 781, 773, 746, 726, 714, 673, 661, 587, 552, 519, 503, 484, 461, 451 and 440 cm⁻¹.

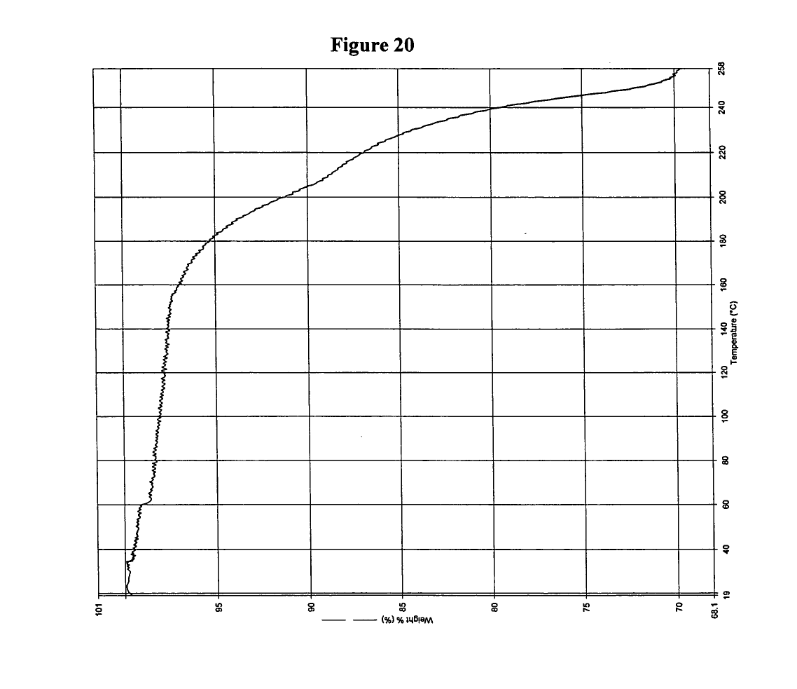

Still other characterizing data for Form E of rimonabant hydrochloride according to the present invention as obtained by thermogravimetric analysis (TGA) is shown in FIG. 20, and it provides characteristic TGA thermogram of anhydrous rimonabant hydrochloride. The loss of weight at 160-260° C. range is mainly due to the decomposition and sublime of crystalline rimonabant hydrochloride at high temperature.

In one favored aspect, Form E of rimonabant hydrochloride provides X-ray powder diffraction (X-RPD) pattern substantially in accordance with FIG. 17 and Table 5.

In another favored aspect, Form E of rimonabant hydrochloride provides differential scanning calorimetry (DSC) thermogram substantially in accordance with FIG. 18.

In still another favored aspect, Form E of rimonabant hydrochloride provides infrared spectrum (IR) substantially in accordance with FIG. 19.

In a further favored aspect, Form E of rimonabant hydrochloride provides thermogravimetric analysis (TGA) thermogram substantially in accordance with FIG. 20.

The present invention encompasses Form E of rimonabant hydrochloride isolated in pure form or in a mixture as a solid composition when admixed with other materials, for example the known forms or solvates (i.e. Form A, B, C, D or amorphous form) of rimonabant hydrochloride or any other material. The content of Form E of rimonabant hydrochloride in a solid composition is over 50% (w/w), preferably 80% (w/w), more preferably over 95% (w/w), and most preferably over 98% (w/w).

Thus in one aspect there is provided Form E of rimonabant hydrochloride in isolated form.

TABLE 5
X-ray diffraction pattern of Form E of rimonabant hydrochloride

| | | FWHM | | Intensity | |
|---|---|---|---|---|---|
| Peak No. | 2θ (deg) | (deg) | d-value | (counts) | I/I₀ |
| 1 | 9.58 | 0.212 | 9.2242 | 55 | 8 |
| 2 | 10.38 | 0.165 | 8.5150 | 96 | 13 |
| 3 | 12.18 | 0.188 | 7.2604 | 293 | 40 |
| 4 | 14.44 | 0.188 | 6.1287 | 743 | 100 |
| 5 | 15.14 | 0.188 | 5.8469 | 136 | 19 |
| 6 | 17.14 | 0.188 | 5.1689 | 74 | 10 |
| 7 | 17.68 | 0.188 | 5.0122 | 97 | 14 |
| 8 | 19.14 | 0.188 | 4.6330 | 103 | 14 |
| 9 | 20.76 | 0.306 | 4.2750 | 131 | 18 |
| 10 | 21.20 | 0.212 | 4.1873 | 416 | 57 |
| 11 | 22.14 | 0.212 | 4.0116 | 103 | 14 |
| 12 | 24.20 | 0.165 | 3.6746 | 312 | 44 |
| 13 | 24.52 | 0.188 | 3.6273 | 319 | 43 |
| 14 | 25.76 | — | 3.4555 | 108 | 15 |
| 15 | 26.82 | 0.165 | 3.3212 | 91 | 13 |
| 16 | 27.60 | 0.165 | 3.2291 | 227 | 31 |
| 17 | 28.52 | — | 3.1270 | 130 | 18 |
| 18 | 29.64 | 0.188 | 3.0114 | 102 | 14 |
| 19 | 30.52 | 0.188 | 2.9265 | 81 | 11 |
| 20 | 32.66 | — | 2.7395 | 65 | 9 |
| 21 | 35.10 | — | 2.5544 | 123 | 17 |
| 22 | 39.24 | 0.188 | 2.2939 | 153 | 21 |
| 23 | 41.46 | — | 2.1761 | 93 | 13 |
| 24 | 43.28 | 0.188 | 2.0887 | 120 | 17 |

In a further aspect there is provided Form E of rimonabant hydrochloride in pure form. The pure form means that polymorph Form E is over 95% (w/w), and preferably over 98% (w/w).

In yet a further aspect there is provided Form E of rimonabant hydrochloride in crystalline form.

In a preferable aspect, the particle size of Form E of the present invention has the median value of the volume mean diameter of the particles within the range of 0.01 μm-500 μm, preferably 5-300 μm, and most preferably 50-200 μm. Such particles are better in stability, good material flow characteristics and thus suitable for bulk preparation and formulation advantages.

The X-ray powder diffraction pattern, differential scanning calorimetry (DSC) thermogram and thermogravimetric analysis (TGA) thermogram of Form E of rimonabant hydrochloride is clearly different from those of other polymorphic forms. Therefore, Form E of rimonabant hydrochloride of the present invention is a new crystalline polymorphic form of rimonabant hydrochloride anhydrous.

In another aspect, the present invention provides a process for preparing Form E of rimonabant hydrochloride, in which an appropriate organic solvent is used as crystallization or recrystallization media. The organic solvent of the present invention can be methanol, ether solvent or a mixture of methanol and ether solvent in any ratio. In the preferred embodiment of the present invention, the ether solvent is ethyl ether, isopropyl ether, methyl n-butyl ether or methyl t-butyl ether. In the more preferred embodiment of the present invention, the organic solvent is a mixture of ether solvent and methanol with a ratio of 1:0.1-10 (v/v). In the most preferred embodiment of the present invention, the organic solvent is a mixture of ether solvent and methanol with a ratio of 3:1 (v/v). The selected approach leads to Form E of rimonabant hydrochloride.

Specifically, the present invention also provides a process for preparation of Form E of rimonabant hydrochloride. A slurry of starting material, crude rimonabant hydrochloride or any polymorph forms of crystalline rimonabant hydrochloride, which can be obtained according to the procedure in Example 196 of U.S. Pat. No. 5,624,941 or any other procedures, admixed with ether solvent or methanol or a mixture of ether solvent and methanol (3:0.5-2, v/v) is heated, suitably to a temperature in the range of from 35° C. to 85° C., such as 50° C. to 70° C., for example 65° C. until all solid materials are dissolved. The hot solution is allowed to cool down to ambient temperature, and the cooled solution is kept at −10° C.-25° C. for crystallization, preferably at 0-10° C., and more preferably at 5° C. Form E of rimonabant hydrochloride is recovered from the solvent by filtration at a temperature of 10° C. to 20° C. Optionally, the solution mixture is seeded with Form E of rimonabant hydrochloride.

More specifically, the crude starting material, rimonabant hydrochloride, can be dissolved in methanol, ether solvent or a mixture of methanol and ether solvent (3:0.5-2, v/v) at any temperature which provides a suitable rate of formation of the a clear supersaturated solution, generally at an elevated temperature for example at the reflux temperature of the solvent and thereafter crystallizing the required product at low temperature, preferably −10° C. to 25° C., more preferably 0° C. to 10° C. Form E of rimonabant hydrochloride is recovered from the solvent by removing the solvent, e.g., by filtration at a temperature of 10° C. to 20° C. The obtained product is dried under vacuum oven or other driers, at a temperature of 25° C. to 65° C., preferably, 35° C. to 55° C., and most preferably, 40° C. to 45° C. The drying time is from 1 hour to 60 hours, preferably 10-48 hours or until the weight of the product is constant.

It is also possible to prepare Form E of rimonabant hydrochloride directly from Form A of rimonabant hydrochloride. Thus, upon heating Form A of rimonabant hydrochloride, the ethanol solvent in crystalline rimonabant hydrochloride ethanolate is desolvated (evaporated from the crystal lattice), resulting in the formation of anhydrous crystalline polymorph which is Form E of rimonabant hydrochloride.

Therefore, in still another aspect, the present invention provides a process for preparing Form E of rimonabant hydrochloride, in which an appropriate organic solvent is used as crystallization or recrystallization media. The organic solvent of the present invention can be ethanol or a mixture of methanol and ethanol in any ratio. In the preferred embodiment of the present invention, the organic solvent is ethanol. More specifically, the crude starting material, rimonabant hydrochloride, can be dissolved in ethanol at any temperature which provides a suitable rate of formation of the a clear supersaturated solution, generally at an elevated temperature for example at the reflux temperature of the solvent and thereafter crystallizing the required product at low temperature, preferably 0° C. to 25° C., more preferably 5° C. to 15° C. The crystalline rimonabant hydrochloride is recovered from the solvent by removing the solvent, e.g., by filtration at a temperature of 10° C. to 20° C. The obtained product is heated under vacuum oven or other driers, at a temperature of 100° C. to 165° C., preferably, 120° C. to 150° C., and most preferably, 130° C. to 140° C. The drying time is from 1 hour to 40 hours, preferably 5-20 hours or until the weight of the product is constant. The selected approach leads to Form E of rimonabant hydrochloride.

It is also possible to prepare Form E of rimonabant hydrochloride directly from rimonabant free base. The rimonabant free base is converted into rimonabant hydrochloride by reacting it with HCl in ether solvent or ethyl acetate (e.g., isopropyl ether, methyl n-butyl ether or methyl t-butyl ether) at a temperature of 25° C.-50° C. The reaction solution is then cooled down to 0° C.-20° C. to allow precipitating or crystallizing the desired product. The obtained product is washed by ether solvent or ethyl acetate, and dried under vacuum oven at 35-50° C. for 5-20 hours or until the weight of the product becomes constant. The selected approach leads to Form E of rimonabant hydrochloride.

Form E of rimonabant hydrochloride as obtained above is characterized by powder X-ray diffraction pattern, substantially as shown in FIG. 17 and Table 5.

Form E of rimonabant hydrochloride as obtained above is characterized by differential scanning calorimetry (DSC) thermogram, substantially as shown in FIG. 18.

Form E of rimonabant hydrochloride as obtained above is characterized by FT-Infrared spectrum (FT-IR), substantially as shown in FIG. 19.

Form E of rimonabant hydrochloride as obtained above is characterized by thermogravimetric analysis (TGA) thermogram, substantially as shown in FIG. 20.

Starting material rimonabant hydrochloride can be prepared according to known procedures, such as those disclosed in U.S. Pat. No. 5,624,941. The starting material, rimonabant hydrochloride, can be crude or pure, anhydrous, hydrate or solvates, amorphous or crystalline material, or a mixture of amorphous and crystalline material. Preferably, the purity of starting material rimonabant hydrochloride is preferably over 95% (w/w), or more preferably over 98% (w/w).

The stability of crystalline polymorphic forms or solvates of rimonabant hydrochloride of the present invention may be determined using conventional quantitative analytical methods. For example the stability of crystalline polymorph forms or solvates of rimonabant hydrochloride may be determined by using accelerated stability conditions, wherein the test compounds are stored under controlled conditions of temperature and humidity over known periods of time, e.g., 40° C./75% relative humidity. Quantitative analysis of the test compounds by high performance liquid chromatography method or by X-ray powder diffraction pattern, DSC, IR or TGA, against appropriate reference standards before, during and after the storage period allows the stability of the test compound to be determined. The above-described solvates or polymorphic forms (Form A, Form B, Form C, Form D and Form E) of rimonabant hydrochloride of the present invention are physically and chemically stable in solid state.

The crystalline polymorph forms or solvates of crystalline rimonabant hydrochloride may be administered per se or, preferably, as a pharmaceutical composition comprising one or more pharmaceutically acceptable carriers.

Accordingly, the present invention also provides a pharmaceutical composition comprising crystalline polymorph forms or solvates of crystalline rimonabant hydrochloride, and one or more pharmaceutically acceptable carriers.

In one aspect, the present invention also provides a pharmaceutical composition comprising Form A of rimonabant hydrochloride, and one or more pharmaceutically acceptable carriers.

In another aspect, the present invention also provides a pharmaceutical composition comprising Form B of rimonabant hydrochloride, and one or more pharmaceutically acceptable carriers.

In a further aspect, the present invention also provides a pharmaceutical composition comprising Form C of rimonabant hydrochloride, and one or more pharmaceutically acceptable carriers.

In a still aspect, the present invention also provides a pharmaceutical composition comprising Form D of rimonabant hydrochloride, and one or more pharmaceutically acceptable carriers.

In a still further aspect, the present invention also provides a pharmaceutical composition comprising Form E of rimonabant hydrochloride, and one or more pharmaceutically acceptable carriers.

The pharmaceutical composition may, if desired, be in the form of a pack accompanied by written or printed instructions for use.

Usually the pharmaceutical compositions of the present invention will be adapted for oral administration, although compositions for administration by other routes, such as by injection and percutaneous absorption are also envisaged.

Particularly suitable pharmaceutical compositions for oral administration are unit dosage forms such as tablets and capsules. Other fixed unit dosage forms, such as powders presented in sachets, may also be used.

Pharmaceutical compositions as provided by the present invention can be prepared by known procedures using well-known and readily available ingredients. In preparation of pharmaceutical compositions as provided by the present invention, crystalline polymorphic forms or solvates of rimonabant hydrochloride, substantially as hereinbefore described in the present invention, can be mixed with a carrier, diluent or excipient.

Pharmaceutical compositions as provided by the present invention can be in the form of tablets, pills, powders, lozenges, sachets, cachets, elixirs, suspensions, emulsions, solutions, syrups, aerosol, ointments, soft and hard gelatin capsules, suppositories, sterile injectable solutions and sterile packaged powders containing, for example, up to 80% by weight of the crystalline polymorph forms or solvates of rimonabant hydrochloride of the present invention.

Some examples of suitable carriers, excipients, and diluents include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water syrup, methyl cellulose, methyl- and propylhydroxybenzoates, talc, magnesium stearate and mineral oil. The compositions can additionally include lubricating agents, wetting agents, and emulsifying and suspending agents, preserving agents, sweetening agents or flavoring agents.

The pharmaceutical compositions of the present invention may be formulated so as to provide immediate, sustained or delayed release of crystalline polymorph forms or solvates of rimonabant hydrochloride, after administration to the patient by employing procedures well known in the art.

Most suitably the pharmaceutical composition will be formulated in unit dose form. Such unit dose will normally contain an amount of the active ingredient in the range of from 1 to 500 mg, more usually 5 to 100 mg, and more especially 10 to 40 mg.

The present invention accordingly provides the polymorph forms or solvates of crystalline rimonabant hydrochloride, for use as an active therapeutic substance.

Thus in an aspect, the present invention provides Form A of rimonabant hydrochloride for use in the treatment of and/or prophylaxis of cardiovascular risk factors such as obesity and related metabolic risk factors, tobacco dependence and certain complications thereof, which cannabinoid type 1 (CB1) receptor is involved.

In a further aspect, the present invention provides Form B of rimonabant hydrochloride for use in the treatment of and/or prophylaxis of cardiovascular risk factors such as obesity and related metabolic risk factors, tobacco dependence and certain complications thereof, which cannabinoid type 1 (CB1) receptor is involved.

In an another aspect, the present invention provides Form C of rimonabant hydrochloride for use in the treatment of and/or prophylaxis of cardiovascular risk factors such as obesity and related metabolic risk factors, tobacco dependence and certain complications thereof, which cannabinoid type 1 (CB1) receptor is involved.

In a still aspect, the present invention provides Form D of rimonabant hydrochloride for use in the treatment of and/or prophylaxis of cardiovascular risk factors such as obesity and related metabolic risk factors, tobacco dependence and certain complications thereof, which cannabinoid type 1 (CB1) receptor is involved.

In a still further aspect, the present invention provides Form E of rimonabant hydrochloride for use in the treatment of and/or prophylaxis of cardiovascular risk factors such as obesity and related metabolic risk factors, tobacco dependence and certain complications thereof, which cannabinoid type 1 (CB1) receptor is involved.

The present invention further provides a method for the treatment of and/or prophylaxis of cardiovascular risk factors such as obesity and related metabolic risk factors, tobacco dependence and certain complications thereof, which cannabinoid type 1 (CB1) receptor is involved, in a human or non-human mammal which comprises administering an effective, non-toxic, amount of polymorphs or solvates of rimonabant hydrochloride of the present invention thereof to a human or non-human mammal with obesity and related metabolic risk factors, tobacco dependence and certain complications in need thereof.

The present invention also provides a use of Form A, Form B, Form C, Form D or Form E of rimonabant hydrochloride, along with one or more additional agents. Since Form A, Form B, Form C, Form D or Form E of rimonabant hydrochloride is a potent and selective antagonist of cannabinoid type 1 (CB1) receptor, a combination therapy with one or more other agents may be particularly desirable.

In the treatment of and/or prophylaxis of cardiovascular risk factors such as obesity and related metabolic risk factors, tobacco dependence and certain complications thereof, which cannabinoid type 1 (CB1) receptor is involved, Form A, Form B, Form C, Form D or Form E of rimonabant hydrochloride, may be taken in doses, such as those described above, one to six times a day in a manner such that the total daily dose for a 70 kg adult will generally be in the range of from 5 to 200 mg, and more usually about 10 to 50 mg.

In a further aspect the present invention provides the use of Form A, Form B, Form C, Form D or Form E of rimonabant hydrochloride, for the manufacture of a medicament for the treatment of and/or prophylaxis of cardiovascular risk factors such as obesity and related metabolic risk factors, tobacco dependence and certain complications thereof, which cannabinoid type 1 (CB1) receptor is involved.

Having thus described the invention with reference to particular preferred embodiments, those in the art can appreciate modifications to the invention as described and illustrated that do not depart from the spirit and scope of the invention as disclosed in the specification. The following examples are set to illustrate the invention, and aid to understanding the invention, but not intended to, and should not be construed to limit its scope in any way.

### Experimental

Absorption infrared spectroscopy (IR) of crystalline polymorphic forms or solvates of rimonabant hydrochloride were obtained using a Fourier-transform infrared spectrometer (Perkim-Elmer, resolution is 4; scans number is 32)

Thermogravimetric analysis (TGA) measurements were performed in a Pyris I TGA of Perkin-Elmer (TGA7) under nitrogen purge. The sample was heated from 40° C. to 280° C. at a scan rate of 10° C./minute.

DSC measurements were performed in a TA instrument with a sealed pan at a scan rate of 10° C./minute from 40° C. to 280° C. under nitrogen purge.

X-ray powder diffraction (XRPD) data were obtained by ARL X-Ray powder diffractometer model XTRA-030. Scanning range 3-50 deg. 2 theta, continuous scan, rate 3 deg./min. The accuracy of peak positions was defined as +/−0.2 degrees due to such experimental differences as instrumentation and sample preparation etc.

### EXAMPLES

### Example 1

### Preparation of Rimonabant Hydrochloride Salt

A solution of hydrochloric acid (2N hydrochloric acid in methyl n-butyl ether) is added dropwise to a solution of N-piperidino-5-(4-chlorophenyl)-1-(2,4-dichlorophenyl)-4-methyl-3-pyrazolecarboxamide (rimonabant, 8.0 g in 30.0 mL methanol, prepared by the procedure described in example 195 of U.S. Pat. No. 5,624,941) until pH is about 1.0. The reaction solution as kept stirring for about 2 hours at ambient temperature. About 80 mL methyl t-butyl ether was added into above solution to precipitate out rimonabant hydrochloride salt. The solid product was filtered off, washed with methyl t-butyl ether and dried in vacuum oven (40° C., 30 in. Hg vac., 10 hours) to give 8.5 g of product, m.p. 218°-220° C. (dec.).

### Example 2

### Preparation of Rimonabant Hydrochloride Ethanlonate (Form a)

Rimonabant hydrochloride (2.2 g), prepared by the procedure described in U.S. Pat. No. 5,624,941, was completely dissolved in absolute ethanol (30 mL) at boiling. The hot solution was then allowed to cool slowly with gentle agitation. After leaving in a refrigerator at 5° C. for 3 days, the crystalline rimonabant hydrochloride ethanolate was filtered off, washed with cold absolute ethanol and dried in vacuum oven (38° C.-40° C., 30 in. Hg vac., 10 hours) to give 1.9 g of product. The dried product was characterized by x-ray powder diffraction (as shown in FIG. 1), DSC (as shown in FIG. 2), IR (as shown in FIG. 3) and TGA (as shown in FIG. 4), and was confirmed as Form A of rimonabant hydrochloride (rimonabant hydrochloride ethanolate).

### Example 3

### Preparation of Rimonabant Hydrochloride Ethanolate (Form A)

Rimonabant hydrochloride (2.2 g), prepared by the procedure described in Example 1 of this application, was completely dissolved in absolute ethanol (30 mL) at boiling. The hot solution was then allowed to cool slowly with gentle agitation. After leaving in a refrigerator at 5° C. for 3 days, crystalline rimonabant hydrochloride ethanolate formed was filtered off, washed with cold absolute ethanol and dried in vacuum oven (38° C.-40° C., 30 in. Hg vac., 5-10 hours) to give 1.9 g of product. The dried product was characterized by x-ray powder diffraction, DSC, IR and TGA, and was confirmed as Form A of rimonabant hydrochloride (rimonabant hydrochloride ethanolate).

### Example 4

### Preparation of Rimonabant Hydrochloride Dichloromethanate (Form B)

Rimonabant hydrochloride (2.0 g), prepared by the procedure described in Example 1 of this application, was completely dissolved in dichloromethane (30 mL) at boiling. The hot solution was then allowed to cool slowly with gentle agitation. After leaving in a refrigerator at 5° C. for 3 days, the crystalline rimonabant hydrochloride dichloromethanate formed was filtered off, washed with cold dichloromethane and dried in vacuum oven (40° C., 30 in. Hg vac., 5-10 hours) to give 1.5 g of product. The dried product was characterized by x-ray powder diffraction (as shown in FIG. 5), DSC (as shown in FIG. 6), IR (as shown in FIG. 7) and TGA (as shown in FIG. 8), and was confirmed as Form B of rimonabant hydrochloride (or rimonabant hydrochloride dichloromethanate).

### Example 5

### Preparation of Rimonabant Hydrochloride Dichloromethanate (Form B)

Rimonabant hydrochloride (2.0 g), prepared by the procedure described in Example 196 of U.S. Pat. No. 5,624,941, was completely dissolved in dichloromethane (30 mL) at boiling. The hot solution was then allowed to cool slowly with gentle agitation. After leaving in a refrigerator at 5° C. for 3 days, the crystalline rimonabant hydrochloride formed was filtered off, washed with cold dichloromethane and dried in vacuum oven (38° C.-40° C., 30 in. Hg vac., 10 hours) to give 1.5 g of product. The dried product was characterized by x-ray powder diffraction, DSC, IR and TGA, and was confirmed as Form B of rimonabant hydrochloride (or rimonabant hydrochloride dichloromethanate).

### Example 6

### Preparation of Rimonabant Hydrochloride Hemi-Isopropanolate (Form C)

Rimonabant hydrochloride (2.2 g), prepared by the procedure described in Example 196 of U.S. Pat. No. 5,624,941, was completely dissolved in 50 mL tetrahydrofuran/ethyl acetate/isopropyl alcohol (0.5:0.5:1, v/v/v) at boiling. The hot solution was then allowed to cool slowly with gentle agitation. After leaving in a refrigerator at 5° C. for 3 days, the crystalline rimonabant hydrochloride hemi-isopropanolate formed was filtered off, washed with cold isopropyl alcohol and dried in vacuum oven (38° C.-40° C., 30 in. Hg vac., 5-10 hours) to give 1.5 g of product. The dried product was characterized by X-ray powder diffraction (as shown in FIG. 9), DSC (as shown in FIG. 10), IR (as shown in FIG. 11) and TGA (as shown in FIG. 12), and was confirmed as Form C of rimonabant hydrochloride (rimonabant hydrochloride hemi-isopropanolate).

### Example 7

### Preparation of Rimonabant Hydrochloride Hemi-Isopropanolate (Form C)

Rimonabant hydrochloride (2.2 g), prepared by the procedure described in Example 1of this application, was completely dissolved in 35 mL isopropyl alcohol at boiling. The hot solution was then allowed to cool slowly with gentle agitation. After leaving in a refrigerator at 5° C. for 3 days, the crystalline rimonabant hydrochloride hemi-isopropanolate formed was filtered off, washed with cold isopropyl alcohol and dried in vacuum oven (38° C.-40° C., 30 in. Hg vac., 5-10 hours) to give 1.7 g of product. The dried product was characterized by x-ray powder diffraction (as shown in FIG. 9), DSC (as shown in FIG. 10), IR (as shown in FIG. 11) and TGA (as shown in FIG. 12), and was confirmed as Form C of rimonabant hydrochloride.

### Example 8

### Preparation of Form D of Rimonabant Hydrochloride

Rimonabant hydrochloride (2.0 g), prepared by the procedure described in Example 196 of U.S. Pat. No. 5,624,941, was completely dissolved in 35 mL methanol/acetonitrile (1:3, v/v) at boiling. The hot solution was then allowed to cool slowly with gentle agitation. After leaving in a refrigerator at 0°-5° C. for 3 days, the crystalline rimonabant hydrochloride formed was filtered off, washed with cold acetonitrile and dried in vacuum oven (40° C., 30 in. Hg vac., 5-10 hours) to give 1.3 g of product. The dried product was characterized by x-ray powder diffraction (as shown in FIG. 13), DSC (as shown in FIG. 14), IR (as shown in FIG. 15) and TGA (as shown in FIG. 16), and was confirmed as Form D of crystalline rimonabant hydrochloride.

### Example 9

### Preparation of Form D of Rimonabant Hydrochloride

Rimonabant hydrochloride (2.0 g), prepared by the procedure described in Example 1 of this application, was completely dissolved in 35 mL methanol/acetonitrile (1:3, v/v) at boiling. The hot solution was then allowed to cool slowly with gentle agitation. After leaving in a refrigerator at 0°-5° C. for 3 days, the crystalline rimonabant hydrochloride formed was filtered off, washed with cold acetonitrile and dried in vacuum oven (38° C.-40° C., 30 in. Hg vac., 5-10 hours) to give 1.3 g of product. The dried product was characterized by x-ray powder diffraction (as shown in FIG. 13), DSC (as shown in FIG. 14), IR (as shown in FIG. 15) and TGA (as shown in FIG. 16), and was confirmed as Form D of crystalline rimonabant hydrochloride.

### Example 10

### Preparation of Form E of Rimonabant Hydrochloride

Rimonabant hydrochloride (2.0 g), prepared by the procedure described in Example 196 of U.S. Pat. No. 5,624,941, was completely dissolved in 35 mL methanol/isopropyl ether (1:3, v/v) at boiling. The hot solution was then allowed to cool slowly with gentle agitation. After leaving in a refrigerator at 0°-5° C. for 3 days, the crystalline rimonabant hydrochloride formed was filtered off, washed with cold isopropyl ether and dried in vacuum oven (38° C.-40° C., 30 in. Hg vac., 5-10 hours) to give 1.6 g of product. The dried product was characterized by x-ray powder diffraction (as shown in FIG. 17), DSC (as shown in FIG. 18), IR (as shown in FIG. 19) and TGA (as shown in FIG. 20), and was confirmed as polymorph Form E of crystalline rimonabant hydrochloride

### Example 11

### Preparation of Form E of Rimonabant Hydrochloride

Rimonabant hydrochloride (2.0 g), prepared by the procedure described in Example 1 of this application, was completely dissolved in 35 mL methanol/methyl n-butyl ether (1:3, v/v) at boiling. The hot solution was then allowed to cool slowly with gentle agitation. After leaving in a refrigerator at 0°-5° C. for 3 days, the crystalline rimonabant hydrochloride formed was filtered off, washed with cold methyl n-butyl ether and dried in vacuum oven (38° C.-40° C., 30 in. Hg vac., 10 hours) for about 5-10 hours to give 1.7 g of product. The dried product was characterized by x-ray powder diffraction (as shown in FIG. 17), DSC (as shown in FIG. 18), IR (as shown in FIG. 19) and TGA (as shown in FIG. 20), and was confirmed as polymorph Form E of crystalline rimonabant hydrochloride.

### Example 12

### Preparation of Form E of Rimonabant Hydrochloride

Rimonabant hydrochloride (2.2 g), prepared by the procedure described in U.S. Pat. No. 5,624,941, was completely dissolved in absolute ethanol (30 mL) at boiling. The hot solution was then allowed to cool slowly with gentle agitation. After leaving in a refrigerator at 10° C. for 2 days, the crystalline rimonabant hydrochloride was filtered off, washed with cold absolute ethanol and dried in vacuum oven (130° C.-140° C., 30 in. Hg vac., 10 hours) to give 1.7 g of product. The dried product was characterized by x-ray powder diffraction, DSC, IR, and was confirmed as Form E of rimonabant hydrochloride.

### Example 13

Formulation of Tablets Containing Polymorph Form of Crystalline Rimonabant Hydrochloride

There were three major steps involved in manufacturing the tablets: (A) preparation of polymorph form or solvate form (any forms of Form A, B, C, D and E) of crystalline rimonabant hydrochloride granular concentrate; (B) preparation of tablet core; (C) coating the tablet core. The amount of each ingredient included in the formulation is shown in Table 6 (quantity in gram).

### A: Preparation of Polymorph Form or Solvate Form of Crystalline Rimonabant Hydrochloride Granular Concentrate

The following ingredients (quantity in gram) were sifted through a clean screen (typically 0.066″): lactose anhydrous, pregelatinized starch, sodium starch glycolate and microcrystalline cellulose.

The screened materials were transferred into a high shear (high-energy) mixer and blended for ten (10) minutes at 100 rpm. The blended material was granulated with purified water. The wet granules were passed through a screen (typically 0.132″), and dried in a fluid bed drier until loss on drying is less than 0.2-0.5% w/w.

TABLE 6
% Composition of polymorph form of crystalline rimonabant
hydrochloride (30%, w/w) granular concentrate

| | Granular | | |
|---|---|---|---|
| | Concentrate | | |
| | Batch # | | |
| | 1 | 2 | 3 |
| Polymorph form or solvate | 90 | 90 | 90 |
| form of crystalline | | | |
| rimonabant hydrochloride | | | |
| Lactose anhydrous | 150 | 0 | 50 |
| Dicalcium phosphate | | 80 | 100 |
| Sodium starch glycolate | 10 | 10 | 10 |
| Pregelatinized starch | 10 | 60 | 10 |
| Microcrystalline cellulose | 40 | 60 | 40 |
| Purified water* | | | |

*Water was removed during the process

The dried granules were passed a screen (typically 0.039″) and blended using a tumble blender for 10 minutes at 12 rpm.

### B: Preparation of Tablet Core Comprising Polymorph Form or Solvate Form of Crystalline Rimonabant Hydrochloride

The concentrated granules are placed into a tumble blender. About two thirds of the lactose is screened and added to the blender, and blended for ten (10) minutes. The microcrystalline cellulose, sodium starch glycollate, magnesium stearate and remaining lactose are screened and added to the blender. The mixtures are blended together for ten (10) minutes. The blended material was compressed on a Kikusui Libra tablet compression machine to a target weight of 200 mg for the 10 mg, 20 mg, or 30 mg tablets.

### C: Preparation of Coated Tablet Comprising Polymorph Form or Solvate Form of Crystalline Rimonabant Hydrochloride

The tablet cores are then transferred to a tablet-coating machine (pan coater). The tablet bed was pre-heated with warm air (approximately 60° C.). The pan speed

TABLE 7
% Composition of tablet core (quantity, mg per tablet) comprising
polymorph form or solvate form of crystalline rimonabant
hydrochloride

| | Formulation Batch# | | |
|---|---|---|---|
| | 1 | 2 | 3 |
| Dosage strength | 10 mg | 20 mg | 30 mg |
| Polymorph form or | 33.0 | 66.0 | 99.0 |
| solvate form of | | | |
| crystalline rimonabant | | | |
| hydrochloride | | | |
| concentrate granules | | | |
| Lactose anhydrous | 137 | | 51 |
| Microcrystalline | 23 | 127 | 43 |
| cellulose | | | |
| Magnesium stearate | 3 | 3 | 3 |
| Total weight | 196 | 196 | 196 |
| Coating material | 4.0 | 4.0 | 4.0 |
| Total weight of coated | 200.0 | 200.0 | 200.0 |
| tablet | | | |

was adjusted to 5-9 RPM before starting the spray cycle. The spray cycle was activated. The exhaust temperature was maintained between 40° C. and 50° C. throughout the cycle. After the proper amount of solution was applied, the coated tablets were dried for approximately two (2) minutes. Steps were repeated for all pans to coat all tablets in the batch and film coated until the tablet weight has increased by 2.0% to 3.5%. All tablets were packaged in plastic bottles with desiccants, and the bottles were heat sealed, then placed under the stress condition.

### Example 14

### Stability Studies

The stability of polymorph forms or solvate forms (Form A, B, C, D and E) of crystalline rimonabant hydrochloride bulk material and tablets was assessed by storing samples for up to 6 or 12 weeks at 25° C./60% RH or 40° C./75% RH. Changes were monitored using a stability-indicating HPLC method. Results were calculated by normalized peak area (npa). Degradants were identified by comparison of their relative retention times against impurity standards.

(i) Polymorph Forms or Solvate Forms (Form A, B, C, D and E) of Crystalline Rimonabant Hydrochloride Bulk Material

It was found that polymorph form or solvate forms of crystalline rimonabant hydrochloride bulk material is stable with respect to formation of known and unknown degradants for over 12 months when stored under normal conditions of temperature and humidity. Similarly, 3 months stability of these polymorph forms or solvate forms (Form A, B, C, D and E) was demonstrated at elevated temperatures and humidity.

ii) Tablets Comprising Polymorph Forms or Solvate Forms (Form A, B, C, D and E) of Crystalline Rimonabant Hydrochloride

Tablets comprising polymorph form or solvate forms (any forms of Form A, B, C, D and E) stored at 25° C./60% RH for up to 12 months are stable with respect to the formation of known and unknown degradants. Similarly, stability for over 3 months was demonstrated at elevated temperatures and humidity.

We claim:

1. A crystalline rimonabant hydrochloride dichloromethane solvate (Form B).

2. A crystalline rimonabant hydrochloride dichloromethane solvate (Form B) of claim 1, characterized by X-ray powder diffraction pattern substantially similar to that presented in FIG. 5 and Table 2.

3. A crystalline rimonabant hydrochloride dichloromethane solvate (Form B) of claim 1, characterized by a differential scanning calorimetry thermogram substantially similar to that presented in FIG. 6.

4. A crystalline Form B of rimonabant hydrochloride dichloromethane solvate of claim 1, characterized by an infrared spectrum absorption bands substantially similar to that presented in FIG. 7.

5. A crystalline rimonabant hydrochloride isopropyl alcohol solvate (Form C).

6. A crystalline rimonabant hydrochloride isopropyl alcohol solvate (Form C) of claim 5, characterized by X-ray powder diffraction pattern substantially similar to that presented in FIG. 9 and Table 3.

7. A crystalline rimonabant hydrochloride isopropyl alcohol solvate (Form C) of claim 5, characterized by differential scanning calorimetry thermogram substantially similar to that presented in FIG. 10.

8. A crystalline rimonabant hydrochloride isopropyl alcohol solvate (Form C) of claim 4, characterized by an infrared spectrum absorption bands substantially similar to that presented in FIG. 11.

9. A crystalline polymorph Form D of rimonabant hydrochloride.

10. A crystalline polymorph Form D of rimonabant hydrochloride of claim 9, characterized by an X-ray powder diffraction pattern substantially similar to that presented in FIG. 13 and Table 4.

11. A crystalline polymorph Form D of rimonabant hydrochloride of claim 9, characterized by a differential scanning calorimetry thermogram substantially similar to that presented in FIG. 14.

12. A crystalline polymorph Form D of rimonabant hydrochloride of claim 9, characterized by an infrared spectrum absorption bands substantially similar to that presented in FIG. 15.

13. A pharmaceutical composition that comprises rimonabant hydrochloride as an active ingredient wherein rimonabant hydrochloride is present in crystalline polymorph forms or solvates as claimed in any preceding claims and one or more pharmaceutically acceptable carriers, excipients or diluents thereof.

14. A pharmaceutical composition according to claim 10, in which the unit dosage form is tablets, capsules, powder, syrup, solution, suspension or ointment.

15. A process for preparing crystalline polymorph Form D of rimonabant hydrochloride, which comprises following steps:
(a) Dissolving or suspending rimonabant hydrochloric in a mixture of acetonitrile and methanol under heating until the solution becomes clear;
(b) Cooling the resulting solution to ambient temperature;
(c) Recrystallizing the product at −110° C. to 25° C.;
(d) Isolating the product by filtration at 5° C. to 25° C.;
(e) Drying the product at 25° C. to 65° C. to obtain Form D of rimonabant hydrochloride.
